(19) United States
(12) Patent Application Publication
(10) Pub. No.: US 2023/0109697 A1
(43) Pub. Date: Apr. 13, 2023

(54) COMBINED ANTI-CYTOKINE THERAPY TO REDUCE METASTATIC CANCER

(71) Applicant: The Board of Trustees of the Leland Stanford Junior University, Stanford, CA (US)

(72) Inventors: Virginie Baylot, Redwood City, CA (US); Renumathy Dhanasekaran, Stanford, CA (US); Dean W. Felsher, San Mateo, CA (US)

(21) Appl. No.: 17/905,043

(22) PCT Filed: Jan. 13, 2021
(86) PCT No.: PCT/US2021/013211

§ 371 Date: Aug. 25, 2022

Related U.S. Application Data
(60) Provisional application No. 62/994,437, filed on Mar. 25, 2020.

(51) Int. Cl. C07K 16/24; A61P 35/04; C12Q 1/6886; A01K 67/027

(52) U.S. Cl. CPC C07K 16/24; A01K 67/0278; A61P 35/04; C07K 16/244; C12Q 1/6886; A61K 2039/507

(57) ABSTRACT

Compositions and methods are provided for alleviating cancer in a mammal.

### CROSS REFERENCE

This application claims priority to U.S. Provisional Application No. 62/994,437, filed Mar. 25, 2020, which is incorporated herein in its entirety for all purpose.

### BACKGROUND

Invasion and metastasis are the most insidious and life-threatening aspects of cancer. While tumors with minimal or no invasion may be successfully removed, once the neoplasm becomes invasive, it can disseminate via the lymphatics and/or vascular channels to multiple sites, and complete removal becomes very difficult. Invasion and metastases kill hosts through two processes: local invasion and distant organ colonization and injury. Local invasion can compromise the function of involved tissues by local compression, local destruction, or prevention of normal organ function. The most significant turning point in cancer, however, is the establishment of distant metastasis. The patient can no longer be cured by local therapy alone at this point.

The process of metastasis is a cascade of linked sequential steps involving multiple host-tumor interactions. This complex process requires the cells to enter into the vascular or lymphatic circulation, arrest at a distant vascular or lymphatic bed, actively extravasate into the organ interstitium and parenchyma, and proliferate as a secondary colony. Metastatic potential is influenced by the local microenvironment, angiogenesis, stroma-tumor interactions, elaboration of cytokines by the local tissue, and by the molecular phenotype of the tumor and host cells.

Local microinvasion can occur early, even though distant dissemination may not be evident or may not yet have begun. Tumor cells penetrate the epithelial basement membrane and enter the underlying interstitial stroma during the transition from in situ to invasive carcinoma. Once the tumor cells invade the underlying stroma, they gain access to the lymphatics and blood vessels for distant dissemination while releasing matrix fragments and growth factors. General and widespread changes occur in the organization, distribution, and quantity of the epithelial basement membrane during the transition from benign to invasive carcinoma.

### SUMMARY OF THE INVENTION

The present invention provides compositions and methods useful for treating tumor invasion and/or metastasis associated with Twist1 expressing cancers. Twist1, when pathologically overexpressed, contributes to metastasis. It is shown herein that Twist1 expressing results in elicitation of a cytokinome comprising cytokine including CCL2, CCL5, CCL7, CXCL1 and IL13. These cytokines enable recruitment and polarization of macrophages to tumor associated macrophages (TAMs). Combined therapeutic inhibition of cytokines can synergistically block metastasis; for example neutralization of CCL2 and IL13 is shown to be synergistic in blocking Twist1-induced metastasis in vivo. In a synergistic response, for example, the combination of agents can produce an effect that is greater than the effect of either agent administered as a monotherapy, and may be greater than the additive effect of each agent administered as a monotherapy.

In some embodiments a method is provided for reducing metastasis and/or invasion of a Twist1 expressing cancer, the method comprising administering an effective amount of a combination of (i) an inhibitor of a cytokine that induces recruitment of macrophages to a tumor environment, e.g. an inhibitor of CCL2, CCL5, CCL7, CXCL1; etc. and (ii) an inhibitor of a cytokine that polarizes macrophages to an M2 phenotype, e.g. IL-13. It is shown herein that inhibition (neutralization) of CCL2 decreased macrophage migration about 7-fold, while inhibition of CCL5, CCL7 or CXCL1 reduced macrophage migration from about 1-fold to about 2-fold. Inhibition of IL-13 did not affect migration. However, inhibition (neutralization) of IL-13 blocked polarization of macrophages, without changing M1 markers. CCL5 and CXCL1 also have an effect on macrophage polarization.

In some embodiments a method is provided for reducing metastasis and/or invasion of a Twist1 expressing cancer, the method comprising administering an effective amount of a combination of (i) an inhibitor of a cytokine selected from CCL2, CCL5, CCL7, and CXCL1; and (ii) an inhibitor of IL-13. In some embodiments the combination comprises (i) an inhibitor of CCL2 and and (ii) an inhibitor of IL-15. In some embodiments the inhibitor of CCL2 is an anti-CCL2 antibody. In some embodiments the inhibitor of IL-15 is an anti-IL-15 antibody.

In some embodiments the cancer is a solid cancer. In some embodiments the cancer is a carcinoma. In some embodiments the carcinoma is a hepatocellular carcinoma. The methods of the invention can provide for increased overall survival of the individual being treated.

Optionally, in some embodiments the cancer cells are determined to overexpress Twist1. Individuals may be selected for therapy by determining the phenotype or genotype of the cancer cells with respect to Twist1, where an individual selected for therapy comprises a cancer that overexpresses Twist1. Individuals may also be tested for the expression of the cytokinome associated with Twist1, e.g. elevated levels of IL-13 and CCL2, where cancers showing positive expression are selected for treatment.

The agents in the combination are administered concomitantly, i.e. each agent is administered within about 45 days, 30 days, 15 days, 7 days, 3 days, 2 days, 1 day or substantially simultaneously with respect to the other agent(s) in the combination. The agents can be considered to be combined if administration scheduling is such that the serum level of both agents is at a therapeutic level. Administration may be repeated as necessary to reduce metastasis of the cancer.

Also provided is an autochthonous transgenic mouse model for cancer in which (i) an oncogene, e.g. MYC; and (ii) a metastasis promoting protein, e.g. TWIST1, are each operably linked to a conditional promoter. In some embodiments the promoter is a tet promoter. In some embodiment the tetracycline-controlled transactivator protein (tTA) In some embodiments the conditional expression is liver-specific, by linking (i) a myc coding sequence and a tet responsive element (TRE); and linking (ii) a Twist1 coding sequence and a TRE in an animal that contains the tetracycline-controlled transactivator protein (tTA) driven by the liver-enriched activator protein (LAP) promoter. Induction of transgene expression induces hepatic cancer from the cells of the animal, i.e. autochthonous cells. The animal provide a useful model for the development and metastasis of tissue specific cancer.

### BRIEF DESCRIPTION OF THE DRAWINGS

FIG. 1: Twist1 induces spontaneous metastatic progression of MYC driven HCC in vivo. a. Mouse model of MYC induced HCC where MYC is under the control of a tetracycline responsive element (TRE) which contain the tetracycline-controlled transactivator protein (tTA) driven by the liver-enriched activator protein (LAP). Doxycycline (Dox) can be used to induce oncogene expression in adult mice. b. Mouse model of MYC/Twist1 induced HCC which inducibly co-expressed MYC, Twist1 and firefly luciferase. c. Bioluminescent imaging (BLI) confirms in vivo oncogene rapid induction by demonstrating liver specific luciferase expression upon withdrawal of doxycycline. d. Serial cross sectional imaging of MYC- and MYC/Twist1-HCC using MRI scan for the abdomen and CT scan for the lungs demonstrate step-wise tumor progression. Both MYC and MYC/Twist1 mice develop multifocal liver tumors but only the latter develops lung metastases. e. Kaplan Meier survival curves show that MYC/Twist1 mice had significantly shorter survival than MYC mice (**p<0.005) while Twist1 transgenic mice remained healthy. f. Pie charts show incidence of metastasis in MYC and MYC/Twist transgenic mice. g. Comparison of liver weights between MYC and MYC/Twist1 tumor bearing mice and control mice which were kept on Doxycycline throughout (**p<0.005). h. Gross and histopathologic appearance of tumors in MYC transgenic model confirming HCC. Lungs do not show any metastases. MYC/Twist1-HCC have histologic appearance of HCC and lung histology shows metastatic disease. j. Model of MYC driven HCC where the mice develop multifocal liver tumors but do not develop metastases. k. Model of MYC/Twist1 HCC where mice develop stepwise progression of liver tumors to metastatic HCC.

FIG. 2: Twist1 expressing tumors are enriched in protumoral macrophages a. Principal component analysis (PCA) showed that MYC- and MYC/Twist1-HCC. overall had distinct, non-overlapping expression profiles. Volcano plot shows comparative analysis of differentially expressed genes between the MYC-HCC and MYC/Twist1-HCC. b. Ingenuity pathway analysis of differentially expressed genes between MYC/Twist1- and MYC-HCC used to identify top biological processes upregulated in MYC/Twist1-HCC. c. Comparison of relative percentage of monocyte and macrophage subpopulations, derived using CIBERSORT analysis, between MYCand MYC/Twist1-HCC (*p<0.05). d. Comparison of relative abundance of major immune subsets between MYC- and MYC/Twist1-HCC (p=ns). e. Immunohistochemistry staining for F4/80 in MYC and MYC/Twist1 normal liver, primary tumor and lung with quantification in bar graph (*p<0.05, **p<0.005). f. Macrophages were isolated from primary tumors and expression level of M2 markers (CD206, Arg1) and M1 markers (iNOS, CCR2) was compared between MYC- and MYC/Twist1-HCC (*p<0.05).

FIG. 3 Tumor associated macrophages are required for Twist1 induced metastasis a. Experimental scheme- MYC and MYC/Twist1 cells were implanted orthotopically and metastatic burden in liver and lung assessed after 4 weeks. b. Representative BLI imaging, gross organ appearance, histopathology of liver (10X) and lungs (1X) from mice orthotopically implanted with MYC and MYC/Twist1 cells c. Comparative quantification of liver and lung metastatic burden between MYC and MYC/Twist1 orthotopic HCC. (**p<0.005). d. Experimental model of orthotopic MYC/Twist1 -HCC treatment either with control liposomes or clodronate liposomes for 4 weeks for macrophage depletion. e. Representative BLI imaging, gross organ appearance, histopathology of liver and lungs from MYC/Twist1 orthotopic HCC bearing mice treated with either control liposomes or clodronate liposomes. f. Comparative quantification of liver and lung metastatic burden between MYC/Twist1 orthotopic HCC bearing mice treated with either control liposomes or clodronate liposomes (**p<0.005). g. Experimental scheme- MYC and MYC/Twist1 cells were injected intravenously and metastatic burden in lung assessed after 4 weeks. h. Representative BLI imaging, gross organ appearance, histopathology of lungs from mice intravenously injected with MYC and MYC/Twist1 cells. i. Comparative quantification of lung metastatic burden between MYC and MYC/Twist1 intravenously injected HCC. (**p<0.005). j. Experimental model of intravenous MYC/Twist1-HCC treatment either with control or clodronate liposomes for 3 weeks for macrophage depletion. k. Representative BLI imaging, and lung histopathology from MYC/Twist1 intravenous HCC injected mice treated with either control liposomes or clodronate liposomes. l. Comparative quantification of liver and lung metastatic burden between MYC/Twist1 intravenously injected HCC bearing mice treated with either control liposomes or clodronate liposomes (**p<0.005).

FIG. 4 Twist1 regulates the cancer cytokinome to induce macrophage recruitment and polarization a. Experimental scheme- the conditioned media (CM) from MYC or MYC/Twist1 cells was used to treat non polarized macrophages for 48 hours. Following that, macrophage migration or polarization was assessed. b. Transwell macrophage migration across a membrane insert when treated with CM from MYC-cells or MYC/Twist1-cells. c. Morphologic appearance of macrophages treated with CM from MYC- or MYC/Twist1-cells. Expression of M2 markers (CD206, Arg1, CX3CR1) in macrophages treated with CM from MYC- or MYC/Twist1-cells. (*p=0.05). d. Experimental scheme- the CM from MYC- or MYC/Twist1-cells were analyzed using Luminex-plate based multiplex ELISA assay. e. Heatmap showing expression levels of top 5 differentially secreted cytokines in conditioned media of MYC- or MYC/Twist1-cells by ELISA. Second heatmap showing mRNA expression levels of top 5 cytokines between MYC- or MYC/Twist1-cells by qPCR. f. Experimental scheme- Co-culture of MYC/Twist1-cells and macrophages separated by a chamber to evaluate chemotaxis of macrophages towards the cancer cells was performed. Neutralizing antibodies to individual cytokines or control antibody were added to the CM of MYC/Twist1-cells. g. Transwell chamber migration assay of macrophages in the upper chamber toward the MYC/Twist1 cells in the lower chamber. MYC/Twist-cells CM was treated with control antibody or neutralizing antibody to IL13, CCL2, CCL5, CCL7 or CXCL1 respectively. (**p<0.005). h. Experimental scheme- CM of MYC/Twist1 cells treated with control antibody or neutralizing antibody to IL13, CCL2, CCL5, CCL7 or CXCL1 respectively was added to non-polarized macrophages for 48 hours. Macrophage polarization was assessed by qPCR for M2 markers (ARG1, CD2016) and M1 markers (IFNAR2, CCR2) (p<0.005).

FIG. 5 Both CCL2 and IL13 are required to promote Twist1-driven metastasis. a. Experimental scheme- Mice orthotopically transplanted with MYC-HCC cells were treated either with PBS or IL13 or CCL2 or CCL2+IL13 recombinant cytokines for 4 weeks. b. Representative histopathology of lung sections from MYC-HCC bearing mice treated with PBS or CCL2+IL13. c. Histopathology of liver (X and 20X) of MYC-HCC bearing mice treated with PBS or CCL2 or IL13 or CCL2+IL13 and IHC for F4/80 and MYC expression in each group. d. Quantification of number of metastatic nodules in the liver control treated or recombinant cytokine treated MYC-HCC bearing mice. (**p<0.01). e. Quantification of macrophage infiltration in liver by IHC staining for F4/80 of MYC-HCC bearing mice liver treated with PBS or CCL2 or IL13 or CCL2+IL13. f. Histopathology of lung (1X and 20X) of MYC-HCC bearing mice treated with PBS or CCL2 or IL13 or CCL2+IL13 and IHC for F4/80 and MYC expression in each group. g. Quantification of number of metastatic nodules in the lung of control treated or recombinant cytokine treated MYC-HCC bearing mice. (**p<0.01). h. Quantification of macrophage infiltration in lung by IHC staining for F4/80 of MYC-HCC bearing mice liver treated with PBS or CCL2 or IL13 or CCL2+IL13.

FIG. 6 Combined inhibition of CCL2 and IL13 has a synergistic effect on prevention of HCC metastasis a. Experimental scheme- Mice orthotopically transplanted with MYC/Twist1 -HCC cells were treated either with control (ctrl) antibody or anti-CCL2 antibody (ab) or anti-lL13 ab or anti-CCL2+IL13 ab for 4 weeks. b. Representative histopathology of lung sections from MYC/Twist1-HCC bearing mice treated with ctrl ab or anti-CCL2+IL13 ab. c. BLI images, histopathology of liver (1X) and lungs (1X) of MYC/Twist1-HCC bearing mice treated with ctrl ab or anti-CCL2 ab or anti-lL13 ab or antiCCL2+IL1 3 ab and IHC for F4/80 (20X) expression in the liver tumor in each group. d. Quantification of number of metastatic nodules in the liver in MYC/Twist1-HCC bearing mice treated with ctrl ab or anti-CCL2 ab or anti-IL13 ab or anti-CCL2+IL13 ab. (**p<0.01). e. Quantification of number of metastatic nodules in the liver in MYC/Twist1-HCC bearing mice treated with ctrl ab or anti-CCL2 ab or anti-IL13 ab or anti-CCL2+IL13 ab. (**p<0.01). f. Quantification of macrophage infiltration in liver by IHC staining for F4/80 of MYC/Twist1-HCC bearing mice treated with ctrl ab or anti-CCL2 ab or anti-lL13 ab or anti-CCL2+IL13 ab. (**p<0.01).

FIG. 7. TWIST1 predicts poor prognosis, TAM infiltration and pro-TAM cytokines in 33 human cancers a. Overall survival and disease free survival in pan-cancer TCGA cohort of 9598 patients from 33 cancers stratified by median TWIST1 expression, b. Overall survival in GSE364 cohort of HCC patients stratified by median TWIST1 expression, c. Comparison of TWIST1 expression between metastatic lesion, primary lesion with metastasis and primary lesion without metastasis in GSE364 cohort of 77 HCC patients. d. Comparison of relative percentage of monocyte and macrophage subpopulations, derived using Cibersort analysis of pan cancer TCGA cohort of 9598 patients from 33 cancers with tumors stratified as MYC/TWIST1^{High} or MYC/TWIST1^{low} (*p<0.05). e. Comparison of relative percentage of M2 macrophage subpopulation, derived using Cibersort analysis of Liver cancer TCGA cohort of 373 patients with tumors stratified as MYC/TWIST1^{High} or MYC/TWIST1^{low} (*p<0.05). f. Correlation of TWIST1 expression with pro-TAM cytokines and TAM markers in TCGA pan-cancer cohort and HCC cohort. g. Experimental scheme for prospective clinical study of plasma cytokine levels. h. Prospective clinical study of plasma levels of cytokines in patients with cirrhosis (n=10) versus patients with HCC (n=25). (*p<0.05, **p<0.01, ***p<0.001) i. Comparison of plasma level of cytokines between patients with single HCC versus multifocal HCC (*p<0.05, **p<0.01, ***p<0.001).

FIG. 8 a. Immunohistochemistry for glutamine synthetase in MYC- and MYC/Twist1-HCC. b. Immunoblotting of MYC and Twist1 levels in MYC- and MYC/Twist1-HCC. c. Immunofluorescence for MYC and Twist1 expression in liver tumors from transgenic MYC and YC/Twist1 mice. d. IHC for phospho histone 3 (PH3) and cleaved caspase 3 (CC3) with quantification comparing MYC- and MYC/Twist1- HCC tumors. e. Wound healing assay to assess migratory capacity of MYC and MYC/Twist1 cells at 0 hr, 24 hour and 72 hr with bar graph showing quantification. f. Transcriptomic expression of epithelial and mesenchymal markers between MYC- and MYC/Twist1-HCC in primary tumors.

FIG. 9 a. The top biological processes upregulated in genes differentially expressed in MYC/Twist1- versus MYC-HCC in transgenic mouse primary tumor tissue, b. Immunohistochemistry staining for neutrophil marker and CD4 in primary liver tumors from MYCand MYC/Twist1 -HCC. Bar graphs show quantification of IHC staining. c. Conditioned media from macrophages extracted from MYC-HCC or MYC/Twist1-HCC tumors was used to treat either MYC- or MYC/Twist1-HCC cells and wound healing assay was performed. d. Bar graphs shows quantification of percentage wound closure at 24 hours (**p<0.005).

FIG. 10. a. Conditional mRNA expression of MYC and Twist1 in primary cell lines derived from MYC-HCC and MYC/Twist1-HCC via PCR. b. F4/80 immunostaining in orthotopic tumors from MYC-HCC and MYC/Twist1-HCC in normal liver, liver tumor and lungs. c. MYC/Twist1-HCC cells treated with control liposomes or clodronate liposomes in vitro and number of colonies quantified. d. Cleaved caspase 3 activity in macrophages and tumor cells treated with control or clodronate liposomes. e. F4/80 immunostaining in orthotopic tumors from MYC-HCC and MYC/Twist1-HCC in normal liver, liver tumor and lungs after treatment with control liposomes or clodronate liposomes. f. Quantification of IHC staining. g. MYC and F4/80 immunostaining of lung tissue after intravenous implantation of MYC-HCC and MYC/Twist1 HCC.

FIG. 11. a. Quantification of migration of macrophages treated with MYC- or MYC/Twist1 -cells conditioned media. b. Expression of M1 marker genes in macrophages treated with conditioned media from MYC-HCC or MYC/Twist1-HCC. c. Spheroid co-culture assay of MYC/Twist1 -cells and macrophages after treatment with either CCL2 antibody or control antibody. F4/80 staining was performed to quantify number of spheroids with macrophage infiltration (*p<0.05). d. Experimental scheme- Conditioned medium of MYC-HCC cells treated with PBS or recombinant IL13 or CCL2 respectively was added to non-polarized macrophages for 48 hours. Macrophage polarization was assessed by qPCR for M2 markers (ARG1, CD2016) and M1 markers (IFNAR2, CCR2) (**p<0.005).

FIG. 12 a. Histopathology , MYC staining or F4/80 staining or Arg1 staining of lung sections (10X) of orthotopic transplanted MYC-HCC bearing mice treated with CCL2+IL13 recombinant cytokines. b. Histopathology of liver and lungs of mice orthotopically transplanted with MYC-HCC cells treated either with PBS or IL4 recombinant cytokine. c. Quantification of number of metastatic nodules in the liver and lungs of control treated or recombinant IL4 treated MYC-HCC bearing mice. d. MYC and F4/80 staining of lung tissue from mice orthotopically transplanted with MYC-HCC cells treated with recombinant IL4.

FIG. 13 a. Arg1 staining of lung sections (10X) of orthotopic transplanted MYC/Twist1-HCC bearing mice treated with control (ctrl) ab or anti-CCL2 ab or anti-lL13 ab or anti-CCL2+IL13 ab. b. Histopathology of liver and lungs of mice orthotopically transplanted with MYC/Twist1-HCC cells treated either with ctrl ab or anti-IL4 ab. c. Quantification of number of metastatic nodules in the liver and lung of MYC/Twist1-HCC bearing mice treated either with ctrl ab or anti-IL4 ab. d. MYC and F4/80 staining of lung tissue from MYC/Twist1-HCC bearing mice treated either with ctrl ab or anti-IL4 ab.

FIG. 14 a. M2 macrophage infiltration in 33 different cancers from the pan-cancer TCGA cohort based on transcriptome deconvolution analysis using Cibersort. b. CIBERSORT analysis of human HCC TCGA RNAseq dataset to show major immune subset composition. c. Kaplan Meier survival curve of TCGA human HCC stratified by median M2 macrophage infiltration. d. Cox proportional hazard survival analysis of 22 immune compartments on survival of HCC patients in TCGA cohort (n=373). e. Multivariate survival analysis of factors independently influencing survival of HCC patients in TCGA cohort (n=373). f. Correlation of M2 macrophage infiltration with vascular invasion, tumor stage and tumor grade. g. Expression of TWIST1 and five cytokines (CCL2, CCL5, CCL7, CXCL1, IL13) in 4 human HCC cell lines stratified by TWIST1 expression. h. Huh7 transfected with TWIST1 gene leads to development of lung metastasis upon intravenous injection in immunocompromised host mice.

### DETAILED DESCRIPTION OF THE EMBODIMENTS

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. l, Cold Spring Harbor Laboratory (1998); and Harlow and Lane, Antibodies: A Laboratory Manual, Cold Spring Harbor Laboratory; (1988).

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rlgG. The term also refers to recombinant single chain Fv fragments (scFv). The term antibody also includes bivalent or bispecific molecules, diabodies, triabodies, and tetrabodies.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)′₂, a dimer of Fab which itself is a light chain joined to V_{H}—C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

In some embodiments, the cancer is hepatocellular carcinoma. Hepatocellular carcinoma is the most common type of primary liver cancer, with an estimated 23,000 new cases and about 14,000 deaths expected in 2012 in the US. However, it is more common outside the US, particularly in East Asia and sub-Saharan Africa where the incidence generally parallels geographic prevalence of chronic hepatitis B virus (HBV) infection. The presence of HBV increases risk of hepatocellular carcinoma by > 100-fold among HBV carriers. Incorporation of HBV-DNA into the host’s genome may initiate malignant transformation, even in the absence of chronic hepatitis or cirrhosis.

Other disorders that cause hepatocellular carcinoma include cirrhosis due to chronic hepatitis C virus (HCV) infection, hemochromatosis, and alcoholic cirrhosis. Patients with cirrhosis due to other conditions are also at increased risk. Environmental carcinogens may play a role; eg, ingestion of food contaminated with fungal aflatoxins is believed to contribute to the high incidence of hepatocellular carcinoma in subtropical regions.

Diagnosis may be based on AFP measurement and an imaging test. In adults, AFP signifies dedifferentiation of hepatocytes, which most often indicates hepatocellular carcinoma; 40 to 65% of patients with the cancer have high AFP levels (> 400 ?g/L). High levels are otherwise rare, except in teratocarcinoma of the testis, a much less common tumor. Lower values are less specific and can occur with hepatocellular regeneration (eg, in hepatitis). Other blood tests, such as AFP-L3 (an AFP isoform) and des-gamma-carboxyprothrombin, are being studied as markers to be used for early detection of hepatocellular carcinoma. Depending on local preferences and capabilities, the first imaging test may be contrast-enhanced CT, ultrasonography, or MRI. Hepatic arteriography is occasionally helpful in equivocal cases and can be used to outline the vascular anatomy when ablation or surgery is planned.

Various systems can be used to stage hepatocellular carcinoma; none is universally used. One system is the TNM system, based on the following: T: How many primary tumors, how big they are, and whether the cancer has spread to adjacent organs; N: Whether the cancer has spread to nearby lymph nodes; M: Whether the cancer has metastasized to other organs of the body. Numbers (0 to 4) are added after T, N, and M to indicate increasing severity. Other scoring systems include the Okuda and the Barcelona-Clinic Liver Cancer staging systems. In addition to tumor size, local extension, and metastases, these systems incorporate information about the severity of liver disease.

Ablative treatments (eg, hepatic arterial chemoembolization, yttrium-90 microsphere embolization [selective internal radiation therapy, or SIRT], drug-eluting bead transarterial embolization, radiofrequency ablation) provide palliation and slow tumor growth. If the tumor is large (> 5 cm), is multifocal, has invaded the portal vein, or is metastatic (ie, stage III or higher), prognosis is much less favorable (eg, 5-yr survival rates of about 5% or less). Radiation therapy is usually ineffective. Sorafenib appears to improve outcomes; and may be dosed in combination with the anti-cytokine therapy described herein.

Interleukin 13 (IL-13) is a protein that in humans is a cytokine secreted by subtypes of T cells, NK cells, mast cells, basophils, and other cells. It is a central regulator in IgE synthesis, goblet cell hyperplasia, mucus hypersecretion, airway hyperresponsiveness, fibrosis and chitinase up-regulation. Although IL-13 is associated primarily with the induction of airway disease, it also has anti-inflammatory properties, and may induce expression of matrix metalloproteinases (MMPs).

IL-13 shares a multi-subunit receptor with IL-4, which receptor is a heterodimer receptor complex consisting of alpha IL-4 receptor (IL-4Rα) and alpha IL-13 receptor (IL-13R1). Heterodimerization activates both STAT6 and the IRS signaling pathways. IL-13 also binds to IL-13Rα2, which may be a negative regulator of IL-13-induced responses.

Anti-IL-13 antibodies are known in the art and have been tested in clinical trials. Dupilumab is a monoclonal antibody IL-13 and IL-4 modulator that targets the shared receptor of IL-4 and IL-13, IL4Ra. Lebrikizumab (INN) is an lgG4 humanized monoclonal antibody that blocks the signaling pathway through the lL-4Rα/lL-13Rα1 heterodimer by binding soluble IL-13 and preventing its link to the receptor. Anrukinzumab is a humanized anti-IL-13 monoclonal antibody which acts to block the cytokine and prevent the activation of lL-13Rα1 and IL-13Rα2. Tralokinumab is a human interleukin-13-neutralizing monoclonal lgG4 antibody which has been tested in severe uncontrolled asthma, ulcerative colitis and idiopathic pulmonary fibrosis.

The chemokine ligand 2 (CCL2), also referred to as monocyte chemoattractant protein 1 (MCP1), is a small cytokine that belongs to the CC chemokine family. CCL2 recruits monocytes, memory T cells, and dendritic cells to the sites of inflammation.

CCL2 is a monomeric polypeptide, with a molecular weight of approximately 13 kDa, which is primarily secreted by monocytes, macrophages and dendritic cells. CCR2 and CCR4 are two cell surface receptors that bind CCL2. CCL2 has chemotactic activity for monocytes and basophils. It may promote tumor growth by angiogenesis, macrophage infiltration and tumor invasion, and distant metastasis.

Inhibitors of CCL2 are known in the art. For example, agents may be monoclonal antibodies. Carlumab (CNTO 888) is a human lgG1_{K} mAb with high affinity and specificity for human CCL2. Preclinical data suggest carlumab may offer clinical benefit to cancer patients. Another antibody is human anti-CCL2/MCP-1 monoclonal antibody (ABN912)

AZD2423 is a potent orally bioavailable non-competitive, negative allosteric modulator of the CCR2 chemokine receptor. In pre-clinical studies, AZD2423 inhibited MCP-1 induced calcium mobilization and chemotaxis of THP-1 cell line with an IC50 of 4 nM. AZD2423 is highly selective (> 500-fold) for CCR2.

Twist-related protein 1 (TWIST1) also known as class A basic helix-loop-helix protein 38 (bHLHa38) is a basic helix-loop-helix transcription factor that in humans is encoded by the TWIST1 gene. Basic helix-loop-helix (bHLH) transcription factors have been implicated in cell lineage determination and differentiation. The protein encoded by this gene is a bHLH transcription factor and shares similarity with another bHLH transcription factor, Dermo1 (a.k.a. TWIST2). The strongest expression of this mRNA is in placental tissue; in adults, mesodermally derived tissues express this mRNA preferentially. Over-expression of Twist or methylation of its promoter is common in metastatic carcinomas.

Twist is activated by a variety of signal transduction pathways, including Akt, signal transducer and activator of transcription 3 (STAT3), mitogen-activated protein kinase, Ras, and Wnt signaling. Activated Twist upregulates N-cadherin and downregulates E-cadherin, which are the hallmarks of EMT. Moreover, Twist plays an important role in some physiological processes involved in metastasis, like angiogenesis, invadopodia, extravasation, and chromosomal instability. Twist also protects cancer cells from apoptotic cell death. In addition, Twist is responsible for the maintenance of cancer stem cells and the development of chemotherapy resistance.

Twist sequences are publicly available, for example at Genbank the nucleotide refseq has the accession number NM_000474 and the protein Refseq has the accession number NP_000465.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body. Metastasis may be reduced, for example, by decreasing the numbers of cancer cells in an organ or body part which is not directly connected to the organ of the original cancerous tumor by at least about 10%, at least about 20%, at least about 30%, at least about 50%, at least about 75%, at least about 90%, at least about 95%, at least about 99%, or more.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient’s cancer cell, e.g., a sample comprising polynucleotides and/or polypeptides that is obtained from a patient’s cancer cell (e.g., a cell lysate or other cell extract comprising polynucleotides and/or polypeptides); and a sample comprising cancer cells from a patient. A biological sample comprising a cancer cell from a patient can also include noncancerous cells.

The term “diagnosis” is used herein to refer to the identification of a molecular or pathological state, disease or condition, such as the identification of a molecular subtype of hepatocarcinoma, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) inhibiting the disease, i.e., arresting its development; and (b) relieving the disease, i.e., causing regression of the disease.

In particular, treating cancer comprises reducing metastasis and invasiveness of the cancer. For example, the spread of a cancer to sites other then the primary site may be reduced. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a cancer therapeutic drug, anti-cytokine antibodies, etc. means administration of the agents at such time that the agents each will have a therapeutic effect. Such concomitant administration may involve concurrent (i.e. at the same time), prior, or subsequent administration of the agents with respect to the administration of each other. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Endpoints that are based on tumor assessments include DFS, ORR, TTP, PFS, and timeto-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Objective Response Rate . ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

Methods are provided for treating or reducing primary or metastatic cancer, particularly metastatic cancer, e.g. adenocarcinomas, colorectal carcinomas; squamous cell carcinomas; basal cell carcinomas; ovarian cancer, pancreatic cancer, breast cancer; etc., and specifically including hepatocellular carcinoma, in a regimen comprising contacting the targeted cells with a combination of agents that (i) inhibits activity of a macrophage recruiting chemokine; and (ii)inhibits activity of a cytokine or chemokine that polarizes macrophages to an M2 phenotype. In some embodiments the inhibiting agent is an antibody, although other agents are known in the art, e.g. the small molecule AZD2423. In some embodiments a combination is administered of an inhibitor of IL-13 activity, which may, for example, bind to IL-13 directly, or bind to an IL-13 receptor, e.g. lL-13Rα, etc.; and an inhibitor of CCL2 activity, which may, for example, bind to CCL2 directly, or bind to a CCL2 receptor, e.g. CCR2, etc.

In some embodiments the cancer cells overexpress Twist1. In some embodiments the gene expression profile of the cancer cells with respect to Twist1 is performed prior to treatment, where a sample, e.g. a biopsy sample, is assesed for expression, and a patient is selected for treatment when there is elevated expression relative, for example to a reference sample, to normal counterpart tissue, to non-metastatis cancers, and the like. Various mathods known in the art for determining gene expression levels may be used, e.g. quantitative PCR, hybridization assays, direct sequencing and the like.

Methods comprise administering to a subject in need of treatment a therapeutically effective amount or an effective dose of the combined agents of the invention, including without limitation combinations of the agents with a chemotherapeutic drug, radiation therapy, anti-tumor antibody, checkpoint inhibitor, CART cell, etc.

Effective doses of the combined agents of the present invention for the treatment of cancer, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of each agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing tumor growth, tumor metastasis or tumor invasion of cancers including carcinomas, etc. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Compositions for the treatment of cancer can be administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient’s condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient’s needs (e.g., Remington’s Pharmaceutical Science (15th ed., 1980) and Goodman & Gillman, The Pharmacological Basis of Therapeutics (Hardman et al., eds., 1996)).

Also within the scope of the invention are kits comprising the compositions described herein and instructions for use. The kit can further contain a least one additional reagent, e.g. a chemotherapeutic drug, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to substantially ablate targeted cells, as described above. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### Animals

Transgenic Animals. The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell. Vectors for stable integration include plasmids, retroviruses and other animal viruses, YACs, and the like. The transgenic animal may be a mouse, rat, rabbit, etc. In some embodiments the animal is a mouse, which may be any of the many inbred or out bred mouse strains known in the art, e.g. B6, Balb/c, etc., and may be immunocompetent or immunodeficient. Generally the animal will be initially heterozygous, and will be bred to be homozygous.

Provided herein is an autochthonous transgenic mouse model for cancer in which (i) an oncogene, e.g. MYC; and (ii) a metastasis promoting protein, e.g. TWIST1, are each operably linked to a conditional promoter. In some embodiments the promoter is a tet promoter. In some embodiment the tetracycline-controlled transactivator protein (tTA) In some embodiments the conditional expression is liver-specific, by linking (i) a myc coding sequence and a tet responsive element (TRE); and linking (ii) a Twist1 coding sequence and a TRE in an animal that contains the tetracycline-controlled transactivator protein (tTA) driven by the liver-enriched activator protein (LAP) promoter. Induction of transgene expression induces hepatic cancer from the cells of the animal, i.e. autochthonous cells. The animal provide a useful model for the development and metastasis of tissue specific cancer.

Transgenic animals of the invention generally comprise an exogenous nucleic acid sequence present as an extrachromosomal element or more usually stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

Transgenic mice may be generated by injection of the DNA construct into the pronucleus of fertilized oocytes. The transgenic animals and xenografted animals may be used in a wide variety of ways, e.g. in gene discovery; for dissection of HSC growth regulation; for screening assays; and the like.

In some embodiments, a biological sample comprising hepatocarcinoma cells isolated from a transgenic mouse described herein is provided. A sample may be obtained from, for example, liver, metastatic sites, etc. Cells may be isolated from the biological sample, for example by flow cytometry, immunoselection techniques, such as high-throughput cell sorting using flow cytometric methods, affinity methods with antibodies labeled to magnetic beads, such as magnetic-activated cell separation (MACS), biodegradable beads, non-biodegradable beads, antibodies panned to surfaces, including dishes, automated single-cell sorting using dual-beam optical trapping, differential adhesion cell sorting, and micro-fabricated fluorescence-activated cell sorting, and any combination of such methods.

The animals and cells derived therefrom may be used for screening candidate therapies modifiers, i.e. compounds and factors that affect cancer metastasis. A wide variety of assays may be used for this purpose, including immunoassays for protein binding; determination of cell growth, differentiation and functional activity; disease phenotypes, and the like.

Typically the candidate compound will be added to isolated cancer cells and/or transgenic animal, and the response of the cells monitored through evaluation of cell surface phenotype, functional activity, patterns of gene expression, and the like. Depending on the particular assay, whole animals may be used, or cell derived therefrom. Cells may be freshly isolated from an animal, or may be immortalized in culture.

The term “agent” as used herein describes any molecule, e.g. protein or pharmaceutical, with the capability of affecting the biological action cancer growth and metastasis. Generally a plurality of assay mixtures are run in parallel with different agent concentrations to obtain a differential response to the various concentrations. Typically, one of these concentrations serves as a negative control, i.e. at zero concentration or below the level of detection. Screening may be directed to known pharmacologically active compounds and chemical analogs thereof.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, examples will be described to illustrate parts of the invention. It is also understood that the terminology used herein is for the purposes of describing particular embodiments.

### EXAMPLES

### Twist1 Induces a Cytokinome That Is Both Required and Sufficient to Drive Metastasis of Hepatocellular Carcinoma

Twist1 is a transcription factor known to physiologically regulate embryonic cell migration. However, Twist1 when pathologically overexpressed, contributes to metastasis. Here we show, in a novel autochthonous transgenic mouse model, that Twist1 overexpression cooperates with MYC to enable hepatocellular carcinoma (HCC) to metastasize in greater than 90% mice. Twist1 was found to elicit a cytokinome including: CCL2, CCL5, CCL7, CXCL1 and IL13. These cytokines enable recruitment and polarization of macrophages to tumor associated macrophages (TAMs). Systemically administering both CCL2 and IL13 enabled non-metastatic MYC-driven HCCs to now metastasize. Antibody neutralization of CCL2 and IL13 is synergistic in blocking Twist1-induced metastasis in vivo. Analysis of 9483 human samples from 33 different cancers in the pan-cancer TCGA cohort revealed that Twist1 expression is generally associated with poor survival (p=7.6x 10⁻¹²), TAM infiltration (p<0.001) and with pro-TAM cytokine expression (p<10⁻⁵⁰). Finally, in a prospective human clinical study, we found that in 25 HCC patients but not in 10 cirrhosis patients, pro-TAM cytokines: CCL2, CCL5, CCL7, CXCL1 and IL13 were enriched and their expression predicted advanced multifocal tumors. Hence, Twist1 induces a pro-TAM cytokinome, which drives and maintains metastasis.

Our work illustrates that combined therapeutic inhibition of cytokines can synergistically block metastasis. Our results are relevant to at least 33 different types of human cancer, where Twist1, cytokines and macrophages were predictive of clinical outcome. Metastasis can be impeded by targeting Twist1 associated specific cytokines that are required to initiate and maintain metastasis.

Metastasis involves tumor cells acquiring the ability to invade, migrate and colonize distant sites. Metastasis still accounts for 90% of cancer related deaths. Several genes, including Twist1 , have been identified that can elicit metastasis. Twist1 is normally required during embryogenesis for cell migration, however, when pathologically activated it can contribute to metastasis. Twist1 can induce metastasis by promoting EMT, inhibiting apoptosis and maintaining stemness. Considerable studies have shown that metastasis is often associated with recruitment and activation of immune cells. Tumor associated macrophages (TAMs) are a major component of the tumor infiltrating lymphocytes that have been shown to promote angiogenesis, induce EMT, and suppress cytotoxic T cells. Cancer cells are purported to recruit TAMs via secreted cytokines like IL10, CSF-1 and CCL2. However, the mechanisms of cytokine secretion in metastasis remain to be established.

Twist1 is known to regulate cytokine expression and inflammation during embryonic development to maintain normal cellular migration. Twist1 has been shown to suppress proinflammatory cytokine expression during embryonic development allowing mesodermal cells to migrate to their destination. Loss of Twist1 in mouse embryo leads to elevation of proinflammatory cytokines resulting in a lethal systemic inflammatory syndrome. This role of Twist1 in suppressing pro-inflammatory cytokine expression has been further corroborated in the context of chronic inflammation. During cancer progression, Twist1 has been reported to induce CCL2 expression and promote angiogenesis. Thus, there are reasons to consider that Twist1 regulates cytokine expression and innate immunity in cancer.

To study Twist1 and metastasis, we generated a novel transgenic mouse model of HCC, using the Tet System to conditionally express either Twist1 alone, MYC alone (MYC-HCC) or MYC and Twist1 (MYC/Twist1-HCC) in hepatocytes. Here we show that Twist1 alone has no phenotype, MYC-HCC only very rarely metastasize, but MYC/Twist1-HCC almost always metastasize. MYC/Twist1-HCC compared with MYC-HCC show increased expression of cytokines (CCL2, CCL5, CCL7, CXCL1 and IL13) that recruit and polarize tumor associated macrophages (TAMs). Administration of CCL2 and IL13 is sufficient to induce metastasis, while blocking either or together prevents metastasis. Further, we show in 33 types of human cancers that Twist1 correlates with TAMs and associated cytokines and predicts prognosis; and we demonstrate in a prospective study that expression of pro-TAM cytokines is associated with HCC and predicts more aggressive HCC. Hence, Twist1-mediated changes in the cancer cytokinome promote recruitment and polarization of TAMs in the cancer microenvironment which in turn drive metastasis, thereby revealing a novel immune-modulatory role of Twist1 in cancer metastasis.

### Results

Twist1 induces spontaneous metastatic progression of MYC driven HCC in vivo. To study the functions of Twist1 in vivo we generated a transgenic mouse which, through the tet system, conditionally expresses Twist1 in a liver specific manner (LAP-tTA/TRE-Twist1/Luc) by crossing TRE-Twist1/Luc mice which harbored the Twist1 and firefly luciferase (luc) genes under the control of a bidirectional tetracycline responsive element (TRE), with the LAP-tTA mice which contain the tetracycline-controlled transactivator protein (tTA) driven by the liver-enriched activator protein (LAP) promoter. Twist1 transgenic mice (LAP-tTA/TRE-Twist1/Luc) exhibited no disease nor gross or microscopic pathology for as long as 18 months of observation thus demonstrating that Twist1 did not play a role in autochthonous tumorigenesis when overexpressed in the liver.

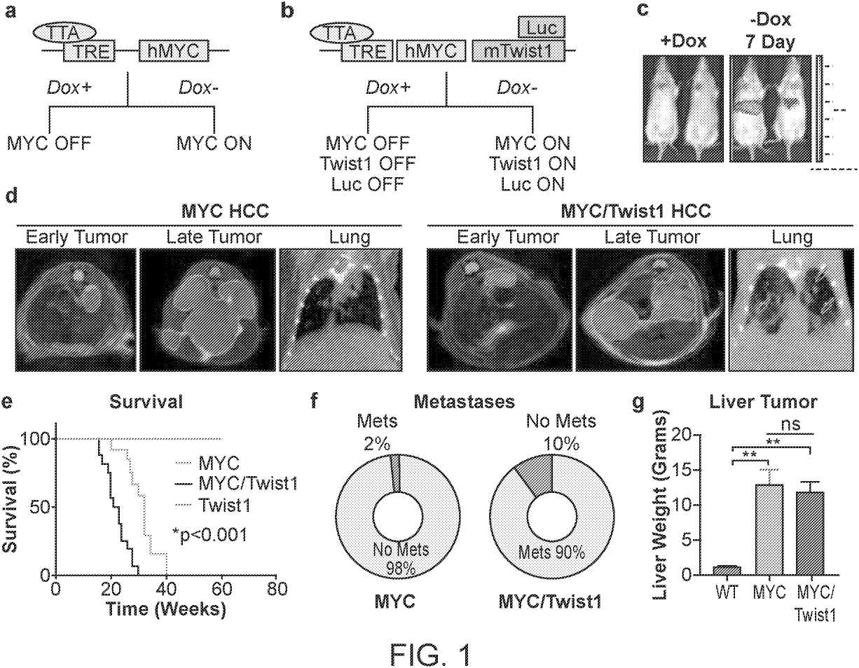

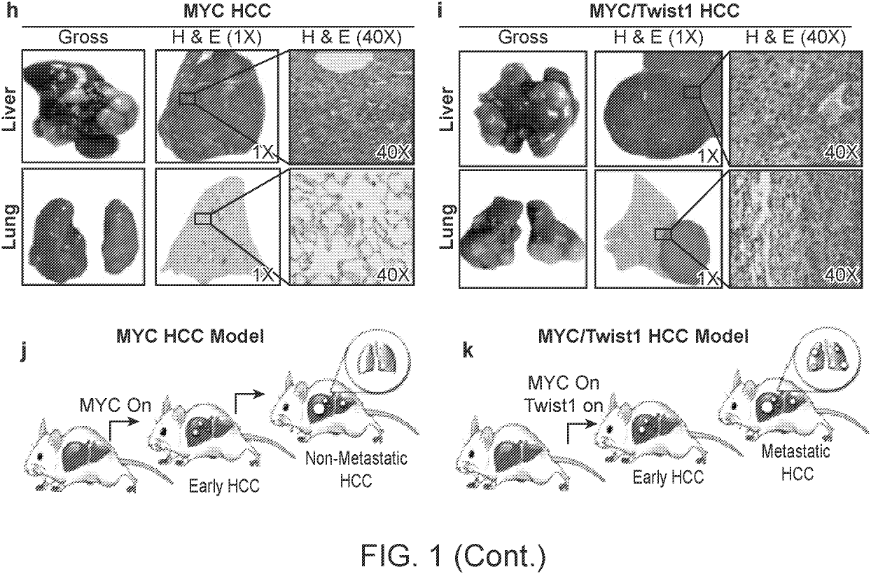

To examine the influence of Twist1 on tumor progression, LAPT-tTA or LAP-tTA/TRE-Twist1/Lucmice were crossed with TRE-MYC (FIG. 1a) to generate transgenic mice that inducibly co-expressed MYC alone (MYC mice) or MYC, Twist1 and luciferase (Luc) in a liver-specific manner (MYC/Twist1 mice) (FIG. 1b). We induced transgene expression in adult mice at 6 weeks of age (FIG. 1b). In vivo Twist1 transgene expression was confirmed to be confined to the liver by measuring the luciferase reporter by bioluminescence imaging (BLI) (FIG. 1c). We followed in vivo tumor progression with serial cross-sectional imaging. Both MYC and MYC/Twist1 mice were observed to develop multifocal liver cancer, while only MYC/Twist1 mice developed lung metastases (FIG. 1d). MYC/Twist1 mice were moribund with HCC sooner and had a median survival of 25 months compared to 32 months in MYC mice (p<0.001, FIG. 1e). MYC mice rarely exhibited metastasis even after extended observation (2%, n=50, FIG. 1f); whereas, MYC/Twist1 mice regularly exhibited rapid onset of metastasis with high penetrance (90%) -metastases to the lungs (70%), peritoneum (60%) and lymph nodes (20%) (n=50, FIG. 1f). Thus, Twist1 combined with MYC expression in liver cells elicits HCC metastasis.

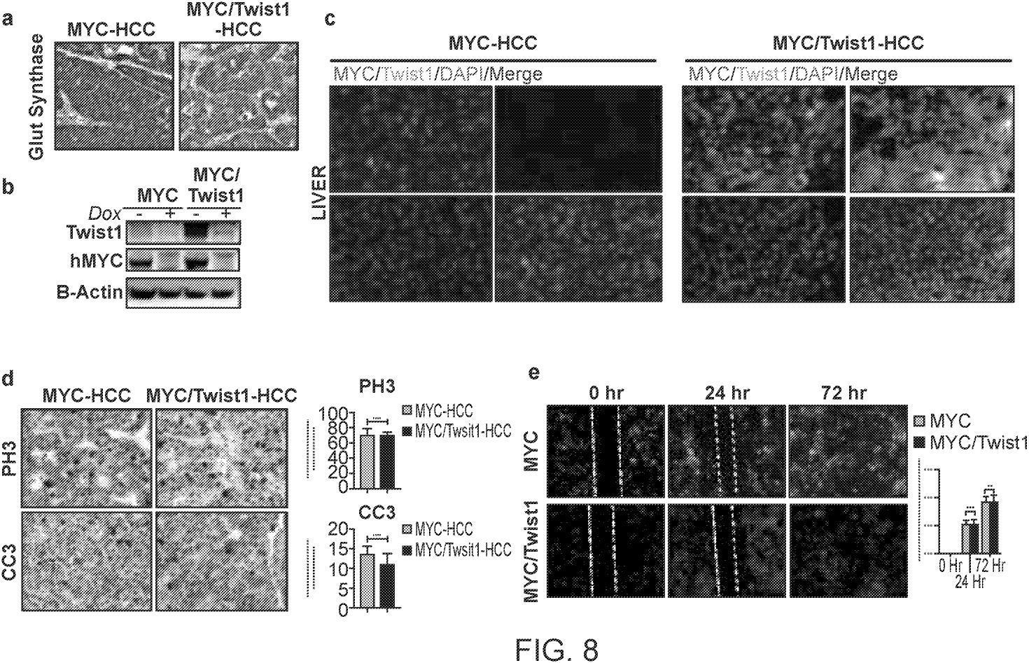

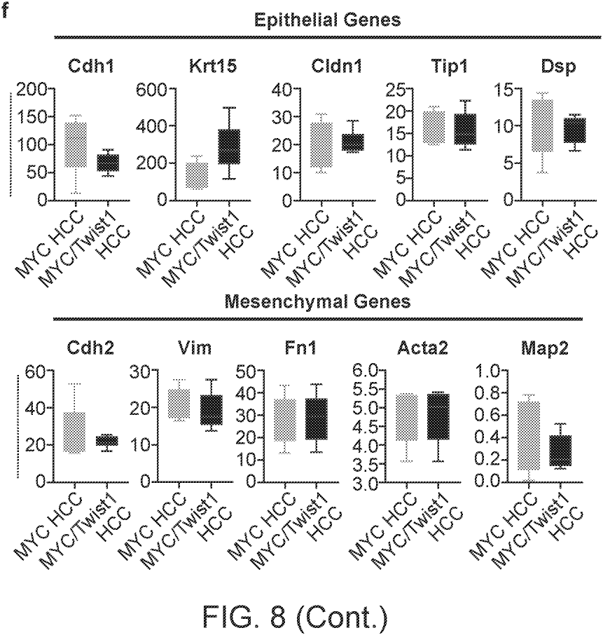

We initially considered a simple explanation for metastasis, as we observed Twist1 was inducing more rapid onset and progression of tumorigenesis. However, the tumor burden in the liver was not statistically different between MYC and MYC/Twist1 mice (FIG. 1g). Also, we did not find any difference in the gross or microscopic appearance of MYC- and MYC/Twist1-HCC (FIGS. 1h-i), which were confirmed to be HCC by a pathologist and by expression of hepatocyte marker glutamine synthetase (FIG. 8a). We considered that Twist1 could be influencing MYC expression levels, but MYC levels were similar between the two tumor models, while Twist1 was only overexpressed in the MYC/Twist1-HCC (FIGS. 8b-1c). We compared tumor cell proliferative index (phospho histone 3 expression) and apoptosis (cleaved caspase 3) between MYC- and MYC/Twist1-HCC and did not find any difference (FIG. 8d). Primary tumor-derived cell lines from MYC- and MYC/Twist1-HCC did not show any difference in migratory capacity (FIG. 8e). Lastly, since Twist1 is a regulator of EMT. We could not find any significant difference in expression of multiple epithelial and mesenchymal markers (FIG. 8f). Therefore, Twist1 drives metastasis of MYC-induced HCC without affecting primary tumor burden, MYC expression, tumor cell proliferation, apoptosis or invasiveness thus demonstrating involvement of non-cell-autonomous mechanisms (FIGS. 1j-k).

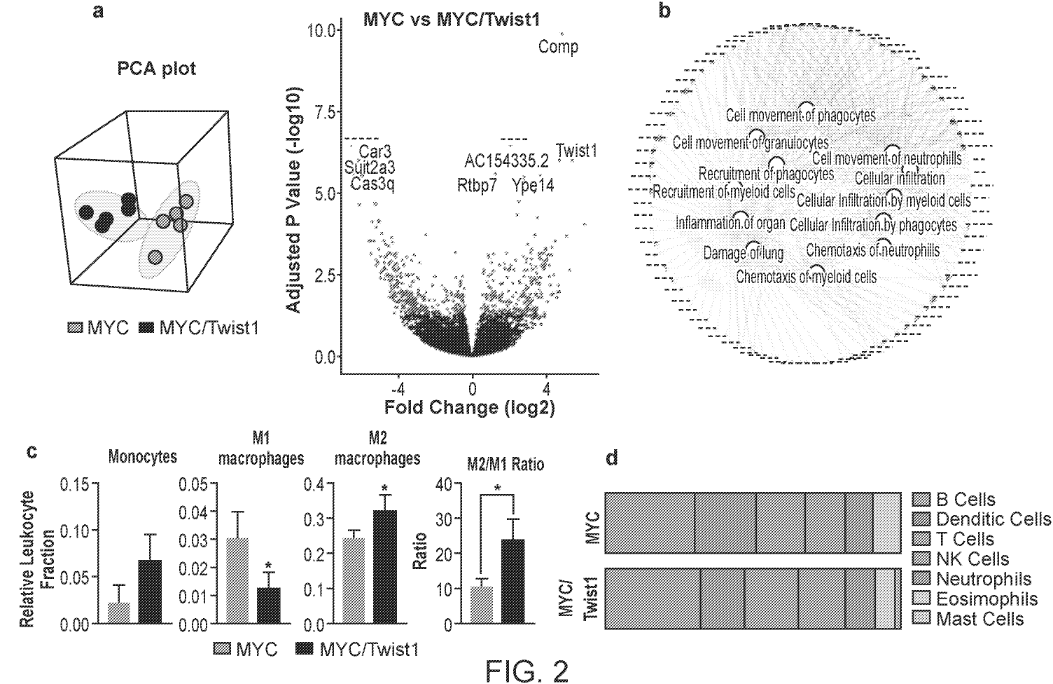

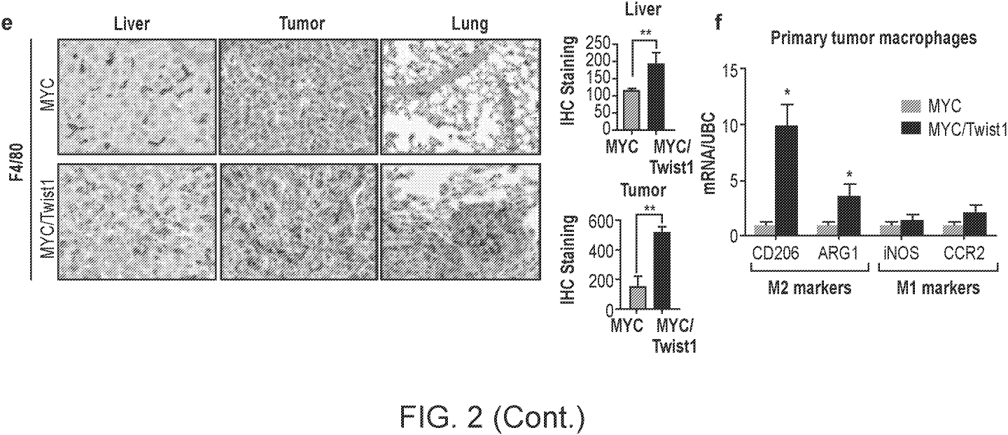

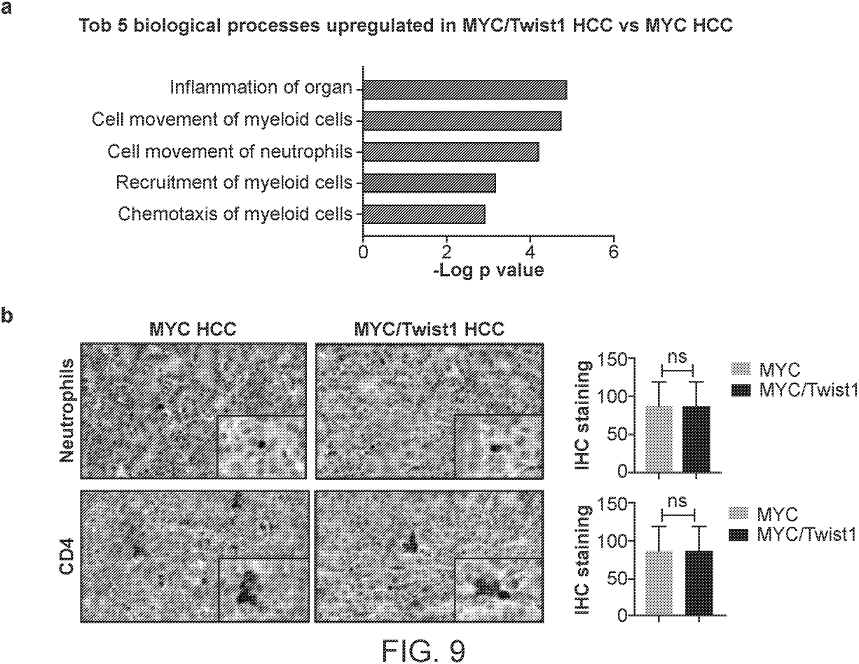

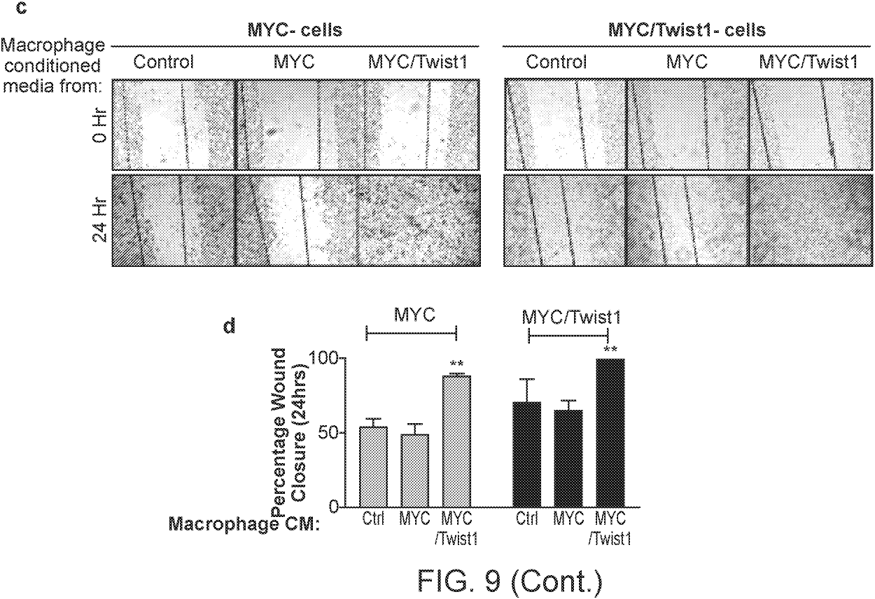

Twist1 expressing tumors have increased tumor associated macrophages. We examined the influence of Twist1 on global gene expression in MYC- and MYC/Twist1-HCC (n=5) using next generation sequencing (NGS) based RNA seq. Through unsupervised hierarchical clustering using principal component analysis (PCA), MYC- and MYC/Twist1-HCC were found to have overall distinct, non-overlapping expression profiles that clustered separately (FIG. 2a). We performed comparative analysis and identified 514 genes (220 up and 294 down) that were differentially expressed between the MYC-HCC and MYC/Twist1-HCC (P<0.001, q<0.05, fold change ≥2) (FIG. 2b). Functional pathway analysis revealed that the top biological processes upregulated in MYC/Twist1-HCC involved inflammatory responses inclded leukocyte infiltration, myeloid cell and granulocyte recruitment (FIG. 2b, FIG. 9a).

CIBERSORT identified M2 macrophages to be significantly enriched in MYC/Twist1 tumors in the 22 immune subsets analyzed (FIG. 2c). MYC/Twist1-HCC exhibited a 15-fold shift in the ratio of M2 to M1 macrophages when compared to MYC tumors (FIG. 2c). No significant difference in other major immune compartments were seen including- B cells, T cells, NK cells, dendritic cells, neutrophils, or mast cells (FIG. 2d). The above findings were confirmed by IHC which demonstrated increased macrophage infiltration in MYC/Twist1 primary and metastatic tumors (FIG. 2e) with no change in neutrophils or CD4 T cells infiltration (FIG. 9b). In addition, TAMs isolated from primary MYC/Twist1-HCC had increased macrophages of the M2 phenotype (CD206^{High}/Arg1^{High}) (FIG. 2f). Conditioned media from TAMs isolated from MYC/twist-HCC but not MYC-HCC increased the invasiveness of both MYC- and MYC/Twist1-HCC tumor cells (FIG. 9c). Hence, Twist1 increases the recruitment and polarization of tumor associated macrophages (M2 phenotype) that produce secreted factors associated with increased cancer cell invasiveness.

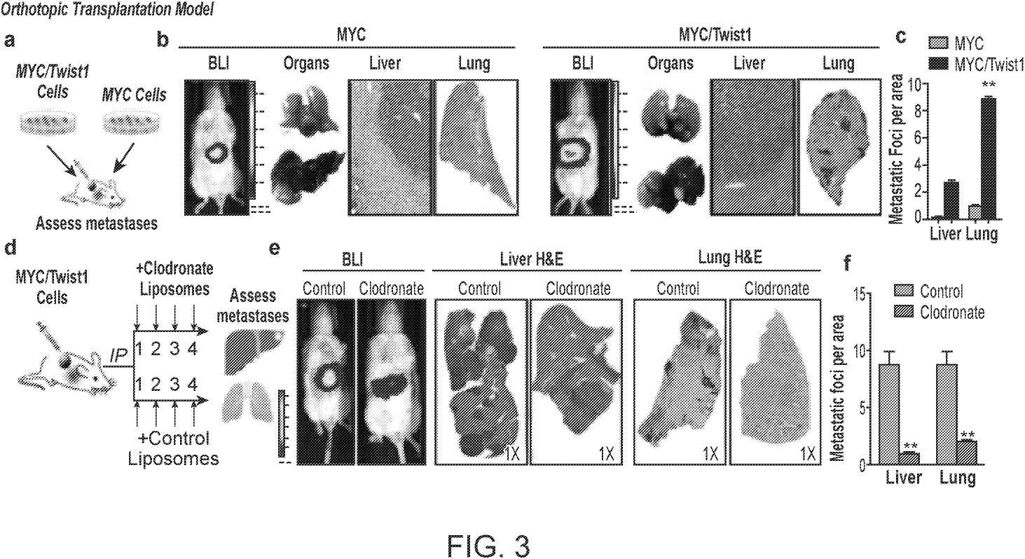

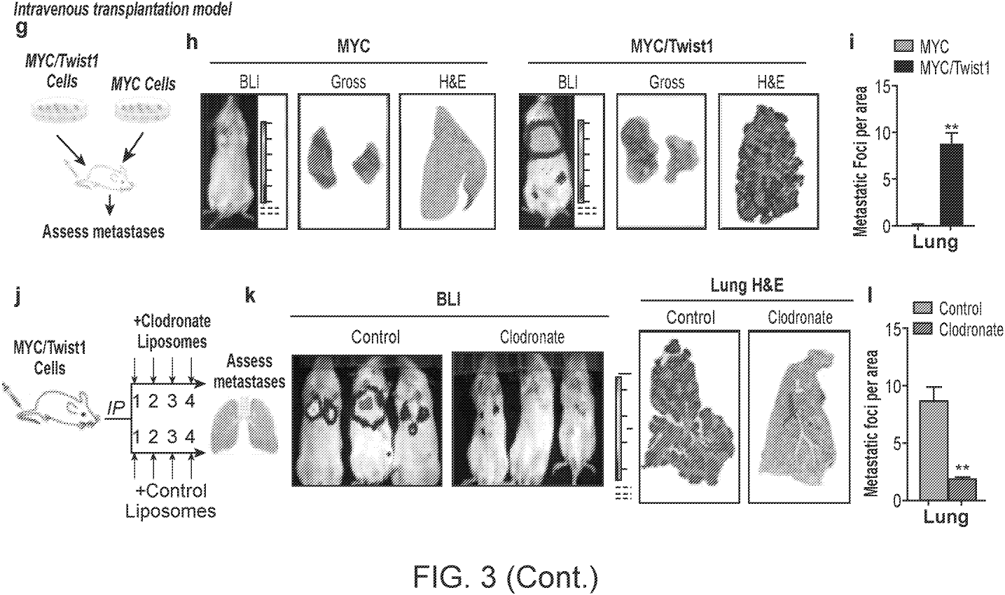

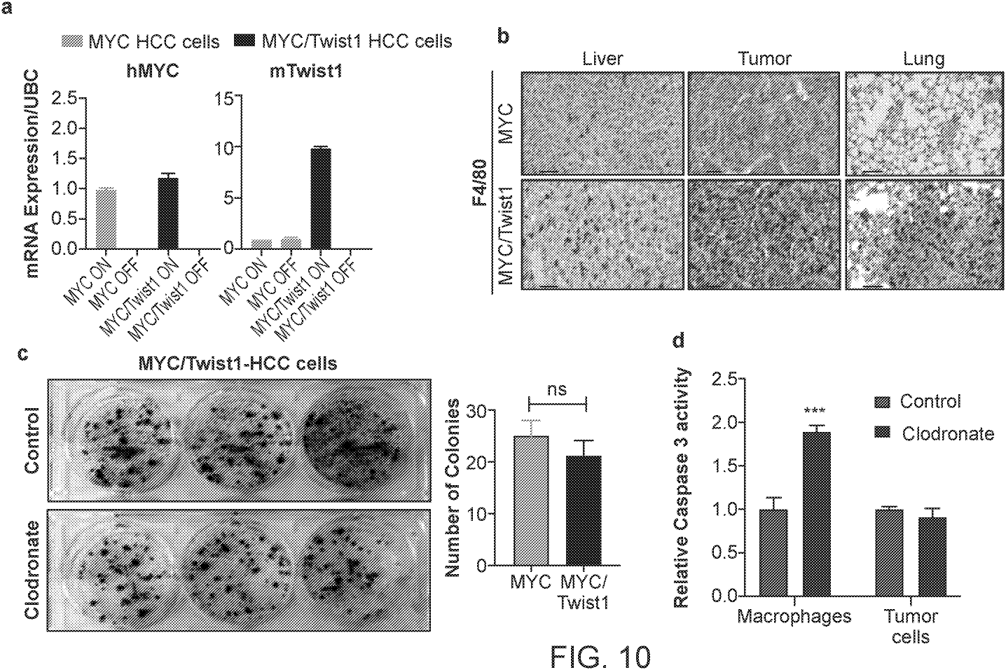

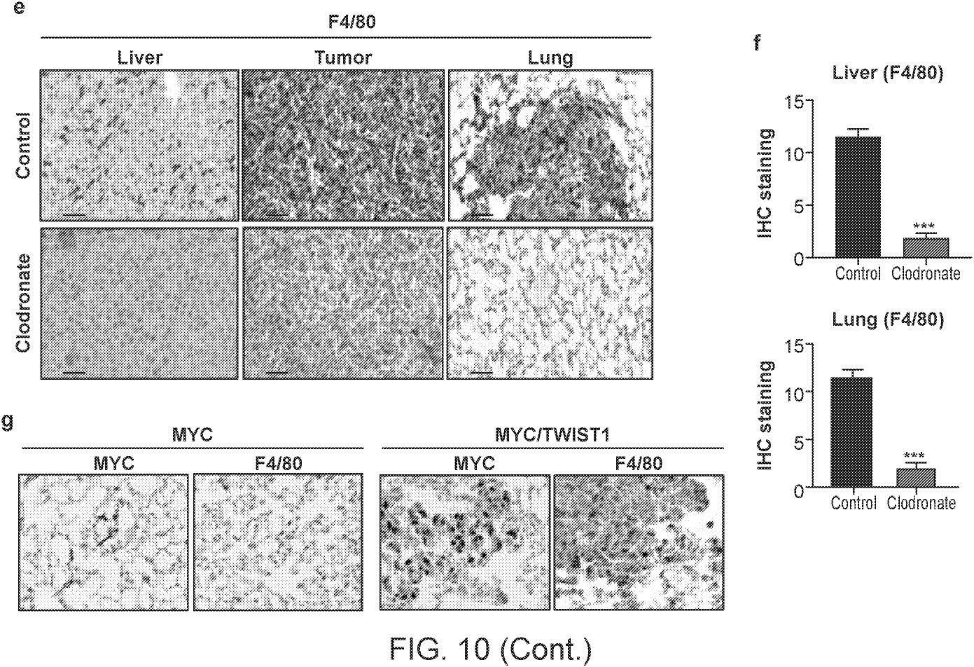

Tumor associated macrophages are required for Twist1 to induce metastasis. We determined if TAMs are required for Twist1 to drive metastasis in vivo. Primary tumor-derived cell lines which conditionally express MYC or MYC/Twist1 (FIG. 10a) were re-introduced in vivo either by orthotopic transplantation into the liver or intravenous injection. Orthotopic implantation (FIG. 3a) of MYC/Twist1- but not MYC-HCC tumor cells in NOD scid gamma (NSG) mice led to pulmonary and intrahepatic metastases with macrophage infiltration (FIGS. 3b-c, FIG. 10b).

Macrophage depletion with clodronate liposomes but not control liposomes in mice orthopically transplanted with MYC/Twist1 —HCC had reduced intrahepatic (p=0.0006, FC 4.4) and lung metastases (p<0.0001, FC 8.8) (FIGS. 3e-f). IHC staining for F4/80 in normal liver, tumor and lungs confirmed a reduction in the number of macrophages (p<0.001) (FIGS. 10e-f). Note, we confirmed that clodronate is toxic to macrophages and not to tumor cells (FIGS. 10c-d). To evaluate if macrophages are required for the colonization step of metastasis, we used the lung trap assay. Intravenous injection of MYC/Twist-HCC but not MYC-HCC cells results in pulmonary metastases associated with with macrophage infiltration (FIGS. 3g-i, FIG. 10g). Clodronate depletion of macrophage reduced pulmonary metastasis (p=0.0003, FC 4.3) (FIGS. 3j-3l). Therefore, Twist1 requires macrophages for invasiveness and colonization steps of metastasis.

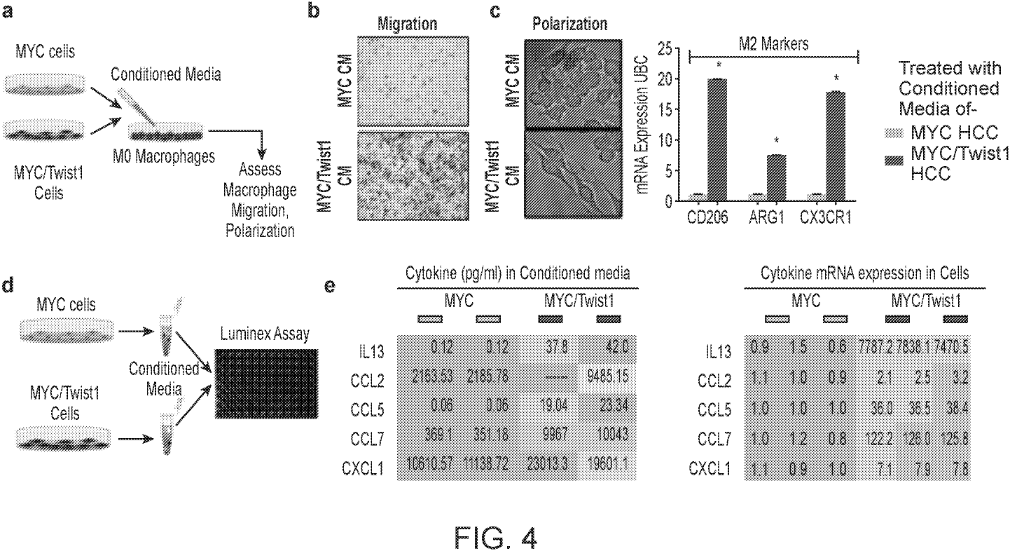

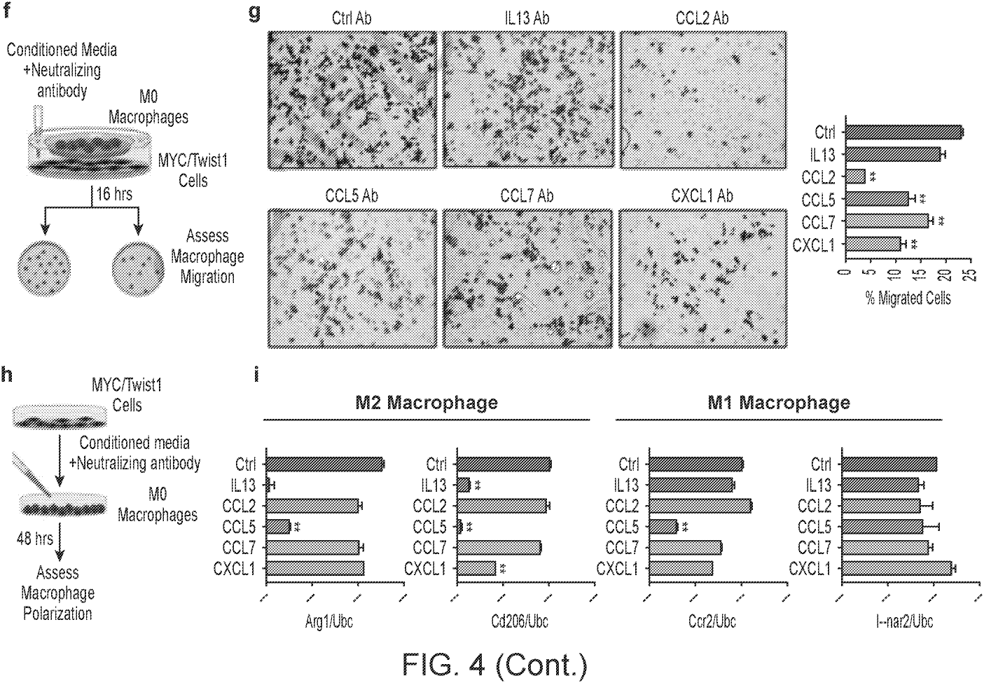

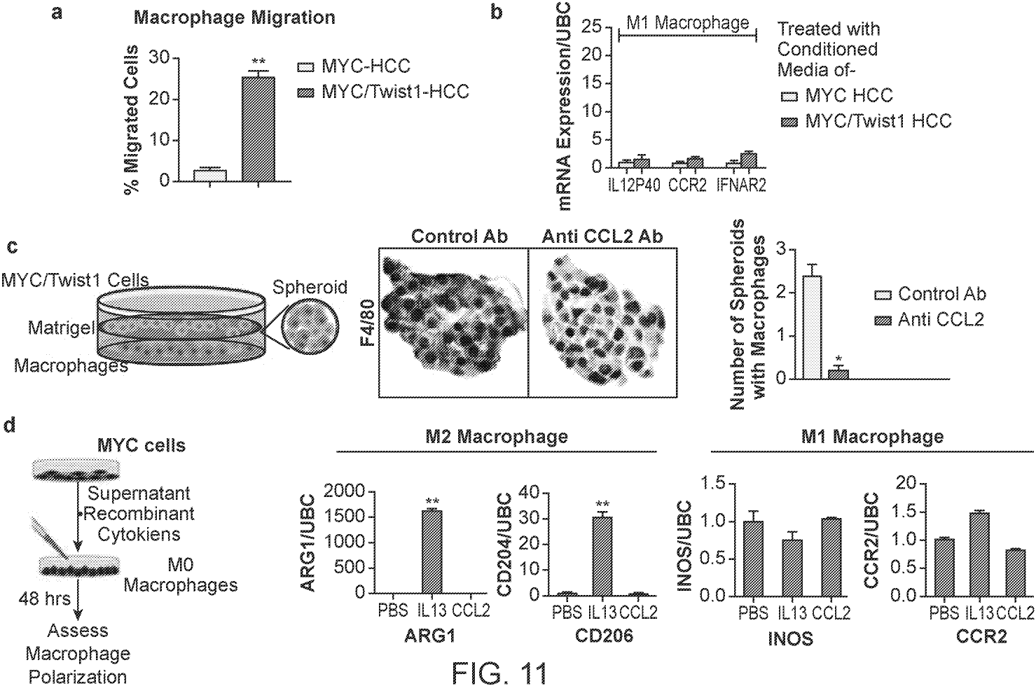

Twist1 regulates cytokines to induce macrophage recruitment and polarization. Our results show that Twist1 causes HCC tumor cells to secrete factors in media that mediate the recruitment of TAMs. Conditioned media derived from MYC/Twist1- or MYC-HCC cells (FIG. 4a) promoted the migration of a macrophage cell line towards cancer cells (FIG. 4b, FIG. 11a). Conditioned media from MYC/Twist1- but not MYC-HCC cells elicited changes in the morphology of macrophages to resemble M2 phenotype (FIG. 4c) and increased expression of M2 markers: CD206, Arg1 and CX3CR1 (FIG. 4c), but not M1 markers: iNOS,CCR2, IFNaR2 (FIG. 11b). Multiplex ELISA for 38 cytokines was performed (FIG. 4d) identifying that MYC/Twist-HCC cells had increased expression of cytokines: IL13, CCL2, CCL5, CCL7 and CXCL1 (p<0.05; Fold change>2, mean>20 ng/ml) (FIG. 4e, FIG. 11a). These 5 cytokines were transcriptionally upregulated in MYC/Twist1 cells compared to MYC cells by qPCR (FIG. 4e).

We assessed the role of cytokines on macrophage recruitment. Antibodies that neutralize CCL2, CCL5, CCL7 or CXCL1 inhibited the ability of conditioned media from MYC/Twist1-HCC to promote migration of macrophages (FIGS. 4f-h). Neutralization of CCL2 decreased migration 7-fold (p<0.0001), CCL5 1.2 fold (p=0.001), CCL7 1.8 fold (p=0.001) and CXCL1 2.1 fold (p=0.0002). Neutralizing IL13 (p=ns, FC 1.2) did not affect macrophage migration. Also, we found that the neutralization of CCL2 inhibited the recruitment of macrophages into MYC/Twist1-spheroids by 3D culture (FIG. 11c). Thus, CCL2 has the biggest effect, although CCL5, CCL7 and CXCL1 also mediate Twist1-induced macrophage recruitment.

We assessed the role of these cytokines on macrophage polarization (FIG. 4h). We found that neutralization of IL13 blocked polarization by 50-fold reduction in CD206 expression (p<0.0001) and 7 fold decrease in Arg1 (p<0.0001) without any change in M1 markers. Neutralization of CCL5 led a 4 fold reduction in CD206 (p<0.001) and 1.1 fold decrease in Arg1 (p<0.05) and CXCL1 led to to 2.4 fold decrease in CD206 (p<0.001) without significant change in Arg1 (p=ns) (FIG. 4i). Adding the cytokines IL13, but not CCL2, to cultured MYC-HCC cells increased M2 marker expression (Supp FIG. 4d). Thus, IL13 has the biggest effect, but also, CCL5 and CXCL1 mediate Twist1-induced macrophage polarization.

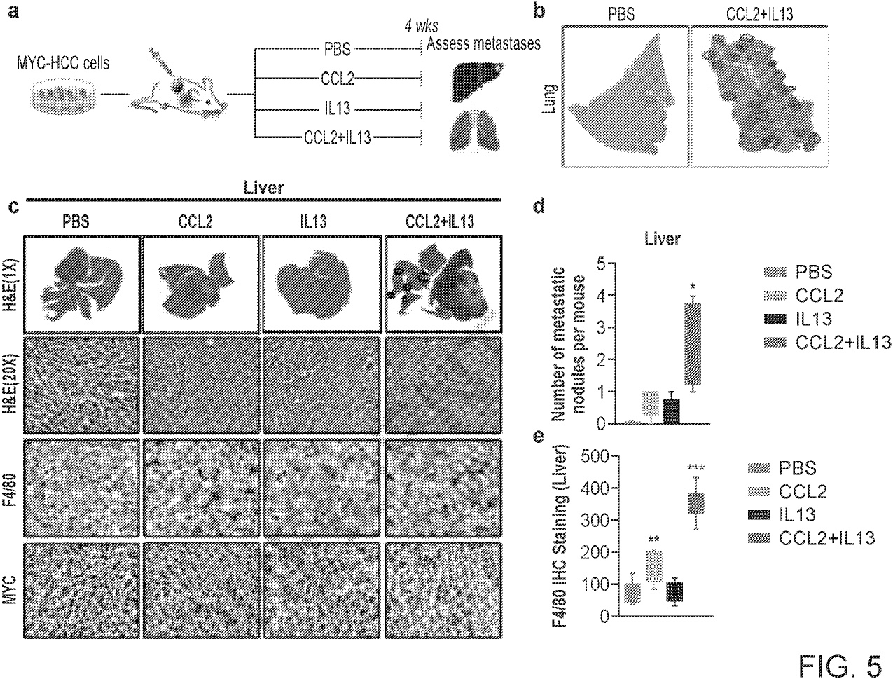

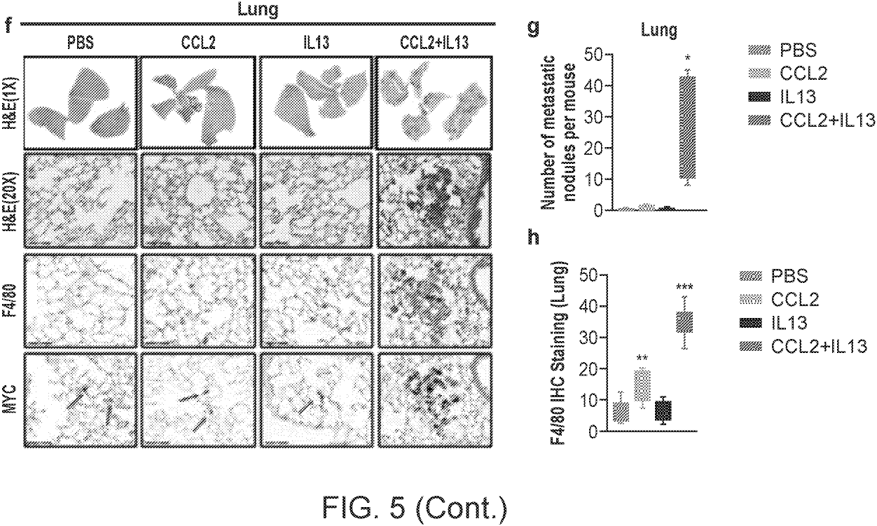

CCL2 and IL13 are required for Twist1-drivenmetastasis. We examined if CCL2 and/or IL13 is sufficient to elicit metastasis. Orthotopic transplants of MYC-HCC in NSG mice were treated either with control, or recombinant CCL2 or IL13 or a combination for 4 weeks (FIG. 5a). Control mice only had rare, small metastatic foci (FIG. 5b). All mice treated with combination IL13 and CCL2 developed multifocal pulmonary (p=0.02, FC=104.4) and few intrahepatic metastases (p=0.008, FC 2.5) (FIGS. 5b-c, FIGS. 5f-g). Treatment of orthotopic MYC-HCC with CCL2 (p=0.390, FC=3) or IL13 (p=0.99, FC=1) did not increase metastases (FIGS. 5c-e, FIGS. 5f-g). Hence, we show that both CCL2 and IL13 are necessary and sufficient to elicit MYC-HCC to metastasize.

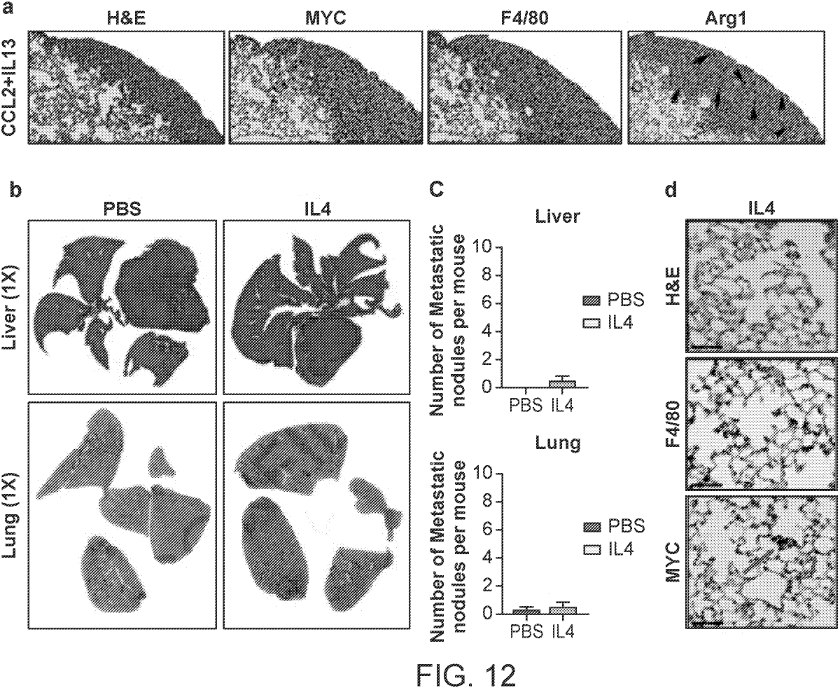

Combination treatment with CCL2 and IL13 increased macrophage infiltration 2.5 fold (p<0.0001) (FIGS. 5e, 5h) and metastatic sites were enriched in Arg1+ TAMs (FIG. 12a). Treatment with CCL2 alone increased macrophage recruitment 1.8-fold (p=0.001) but not metastasis (FIG. 5e). Treatment with IL13 alone did not increase macrophage infiltration (FIG. 5e). As a control, treatment with recombinant IL4, which was not increased in MYC/Twist1-HCC, did not elicit macrophage recruitment or increase metastasis (FIGS. 12b-d). Thus, the combination of CCL2 and IL13 can induce metastasis of MYC-HCC.

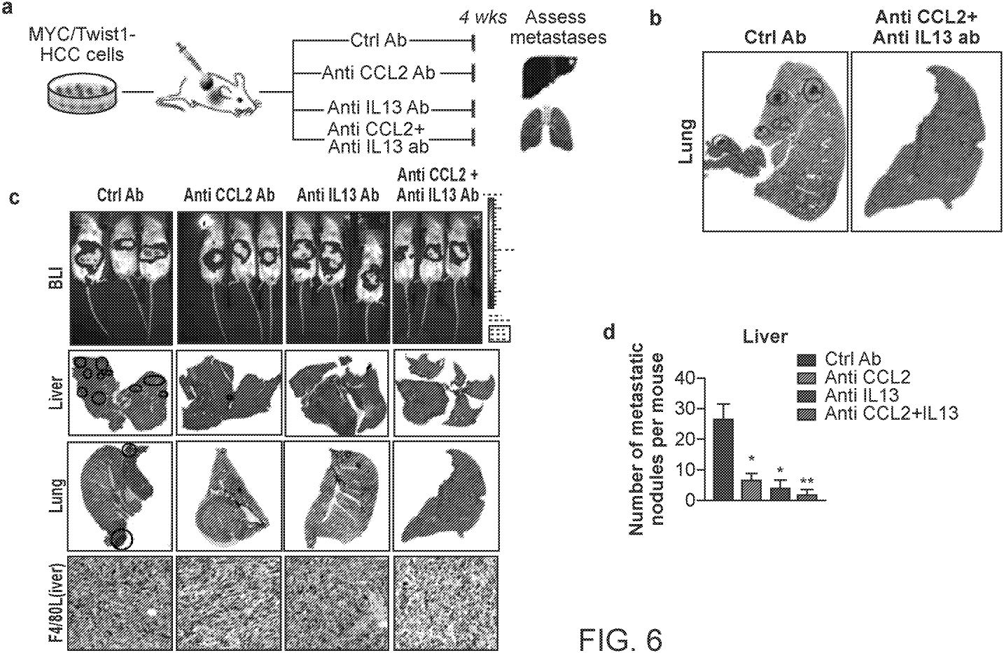

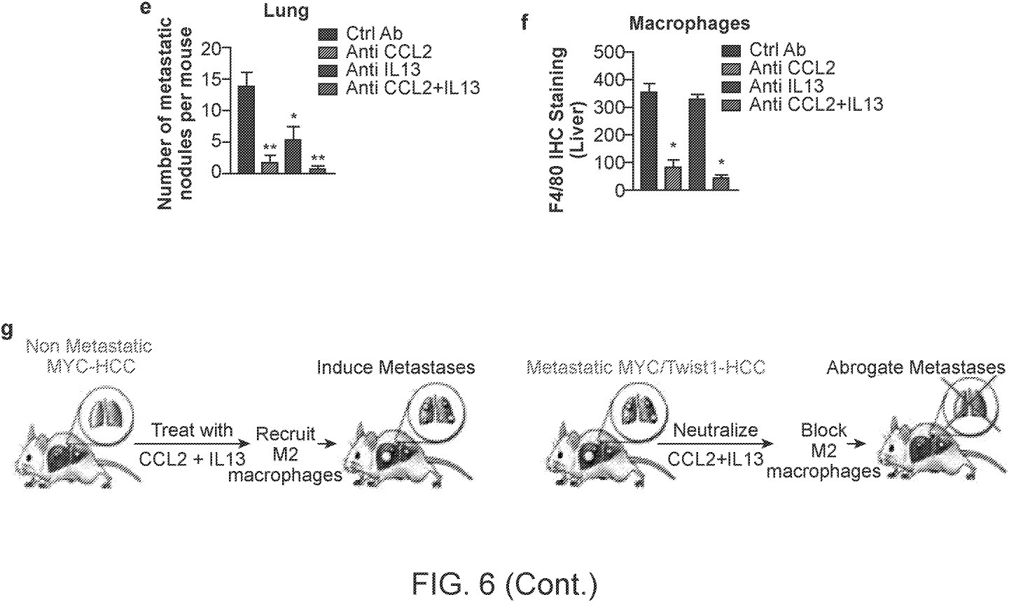

Combined inhibition of CCL2 and IL13 synergistically inhibits metastasis. To examine if CCL2 and IL13 were required for Twist1 to induce metastasis, we performed orthotopic transplants of MYC/Twist1-HCC in NSG mice and treated either with control antibody, anti-CCL2 ab, anti-IL13 ab, or combination treatment anti-IL13 and CCL2 ab, for 4 weeks (FIG. 6a). Control antibody treated mice developed multifocal metastases. Treatment with combination of anti-CCL2 and anti-IL13 ab led to 14-fold decrease in lung metastases (p=0.0009) (FIG. 6b) and a 12-fold decrease in liver metastases (p=0.0006, FIGS. 6c-d). CCL2 inhibition alone led to 7-fold decrease (p=0.002) and IL13 to a 2.5-fold decrease (p=0.03) in lung metastases(FIGS. 6c-6e).

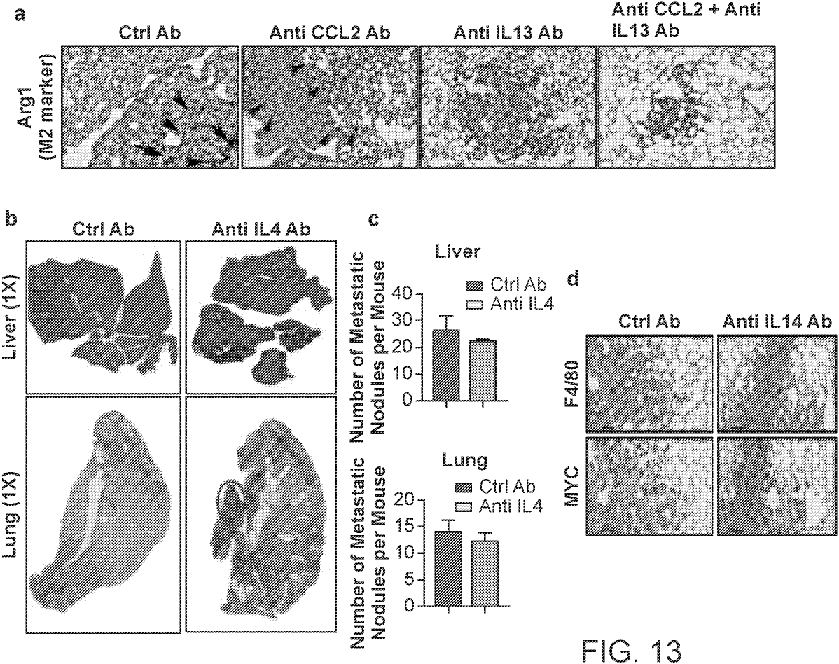

The combined inhibition of CCL2 and IL13 reduced macrophage recruitment and polarization (p<0.0001) (FIGS. 6c, 6f). Inhibition of CCL2 (p<0.001), but not IL13 (p=0.435), decreased macrophage recruitment. Inhibition of IL13 either alone or in combination with CCL2 led to loss of Arg1+ M2-like macrophages at metastatic sites (FIG. 13a). Thus, inhibition of CCL2 decreases macrophage recruitment and IL-13 macrophage polarization. As a control, anti IL4, as expected, had no effect on metastasis (FIGS. 13b-c). The combined inhibition of CCL2 and IL13 synergistically reduced metastases. CCL2 and IL13 are both sufficient and required for Twist1 induced metastases (FIG. 6g).

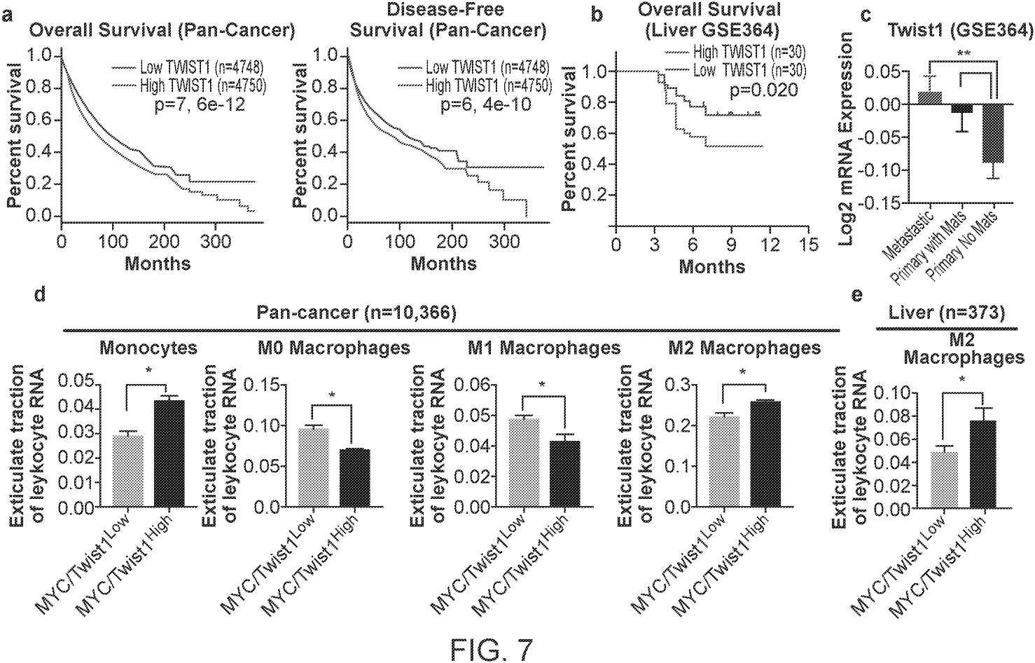

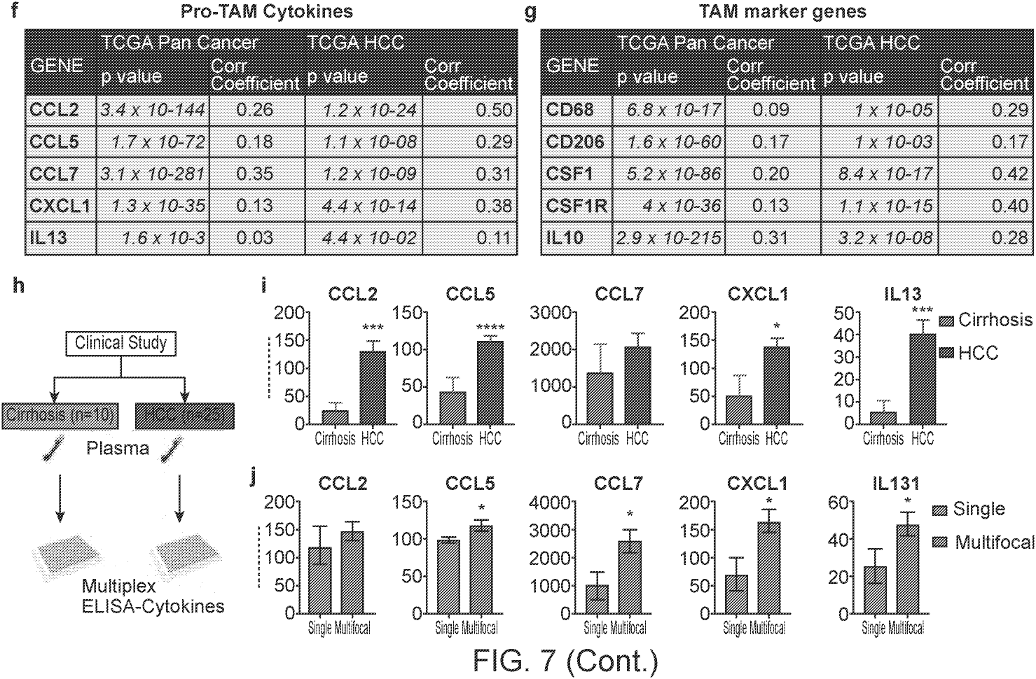

TWIST1 predicts poor prognosis, TAM infiltration and pro-TAM cytokines in 33 human cancers. We evaluated the prognostic role of TWIST1 in 33 human cancers from 9458 patients profiled in the TCGA pan-cancer study. Higher TWIST1 expression was associated with significantly poor overall survival (OS) (p=7.62×10⁻¹², HR 1.3) and also significantly poor disease free survival (DFS) (p=6.4×10⁻¹⁰, HR 1.7) (FIG. 7a). In the TCGA liver cancer dataset, TWIST1 expression was associated with poor survival only in the highest quartile (p=0.036, HR 1.7); however, this cohort had mostly early stage tumors. In another cohort of 144 patients with metastatic HCC, TWIST1 predicted poor survival (p=0.020, HR 1.9; FIG. 7b) and metastasis (FIG. 7c). Thus, TWIST1 predicts survival in all human cancers, and specifically in liver cancer.

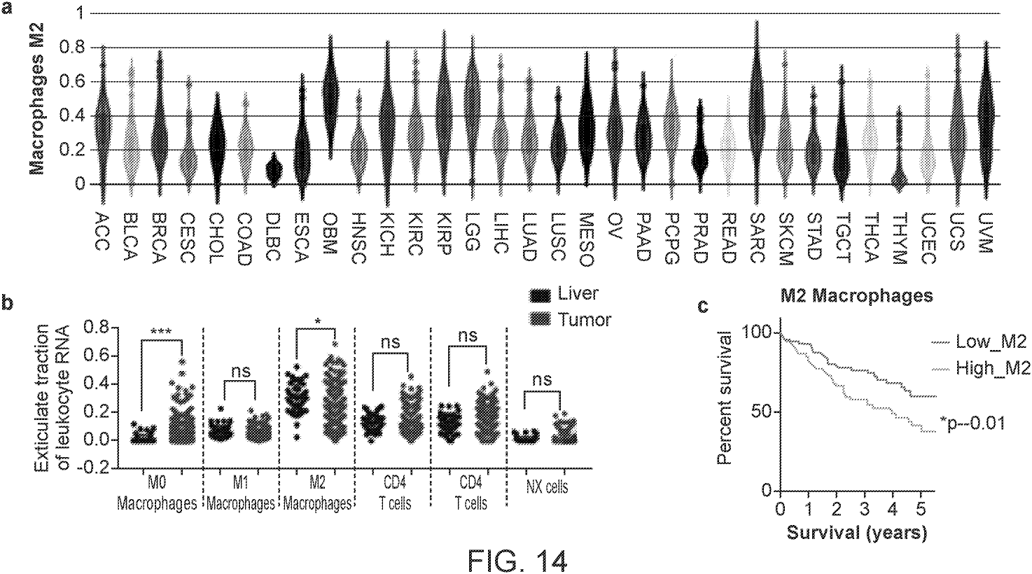

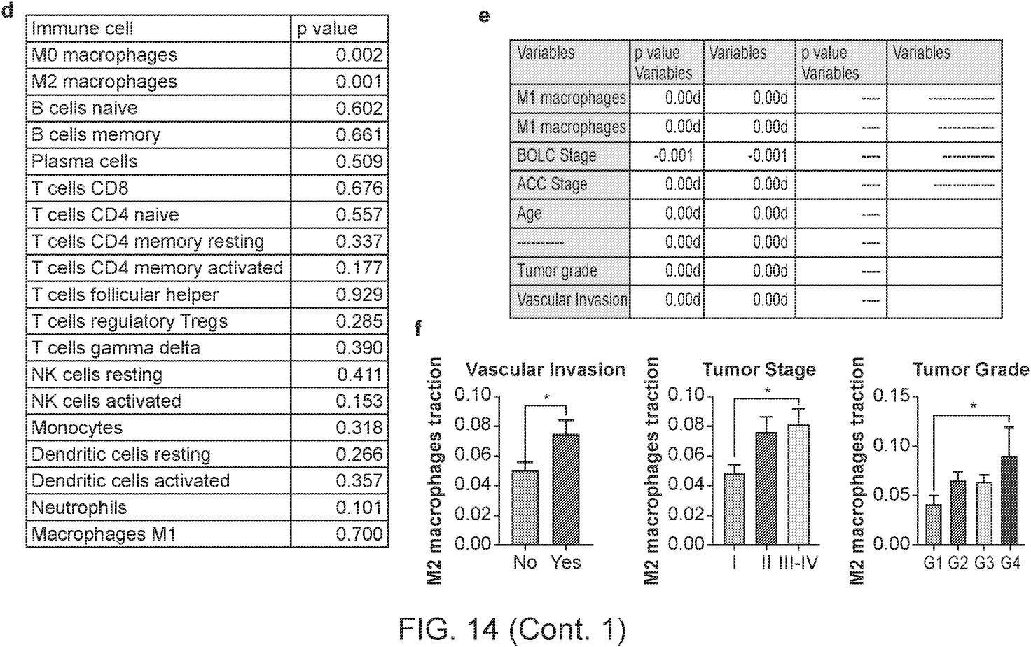

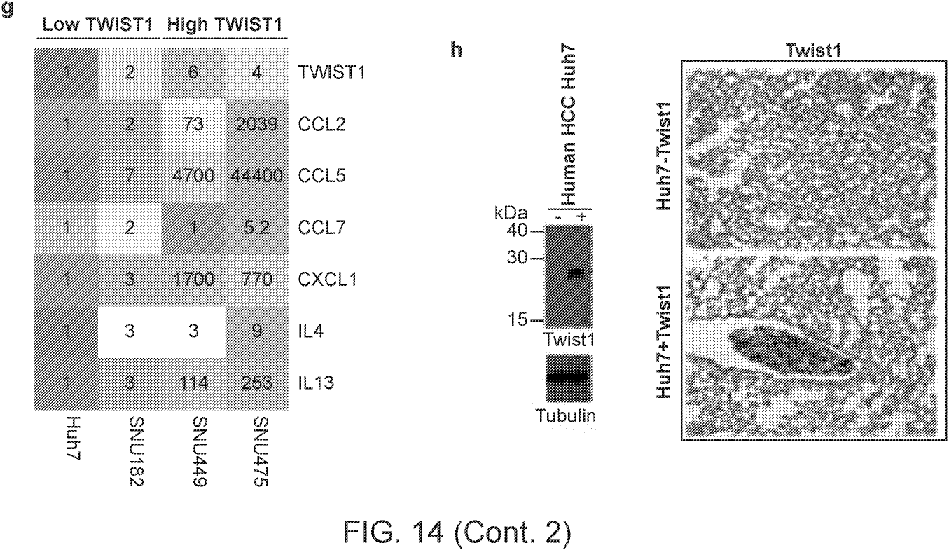

To study the influence of TWIST1 on immune populations within human tumors, we performed CIBERSORT analysis of 10,366 tumors from the human pan-cancer TCGA study. TAMs were the most common infiltrating immune cells in most types of human cancers (FIG. 14a) including HCC (FIG. 14b). The pan-cancer TCGA tumors were organized into two groups based on the expression profile concordant with our mouse model and found that compared to MYC/TWIST1 Low tumors, MYC/TWIST1 High tumors were infiltrated with significantly higher proportion of monocytes (29% vs 44%; p<0.001) and M2 macrophages (22% vs 26%, p<0.001), while there was a lower proportion of M0 (10% vs 7%, p<0.001) and M1 macrophages (6% vs 4%, p<0.001) (FIG. 7d). This was true for the HCC cohort (M2 macrophages 25% vs 29%, p=0.004; FIG. 7e). Moreover, TWIST1 expression correlated with M2 macrophage related genes 36 both in the pan-cancer TCGA data and HCC (FIG. 7f). Increased TAM infiltration was highly prognostic of overall survival on univariate and multivariate analysis (p=0.01, HR 16.0, FIGS. 14c-14e). TAM was associated with presence of vascular invasion, advanced stage and poor tumor grade (FIG. 14f). Thus, TWIST1 predicts TAM infiltration in many human cancer types, including HCC, which in turn is associated with poor clinical outcome.

We examined if TWIST1 expression was associated with pro-TAM cytokines in human tumors. TWIST1 correlated with the pro-TAM cytokines CCL2, CCL5, CCL7, CXCL1 and IL13, both in the pan-cancer TCGA data and HCC (FIG. 7g). TWIST1 was measured in 4 human HCC cell lines. Two cell lines with higher TWIST1 expression were associated with higher CCL2, CCL5, CCL7, CXCL1 and IL13 when compared to TWIST1^{Low}cell lines (FIG. 14g). Further, the expression of TWIST1 via retroviral expression vector introduction in the human HCC cell lines, Huh7, elicited pulmonary metastases in NSG mice (FIG. 14h). Thus, TWIST1 predicts pro-TAM cytokine expression and metastasis in human HCC.

Lastly, we performed a prospective study to examine if TWIST1 associated pro-TAM cytokines were expressed in human patients with HCC (n=25) but not in patients with cirrhosis of the liver (n=10)(FIG. 7h). We found CCL2, CCL5, CXCL1 and IL13, were significantly elevated in the plasma of patients with HCC (FIG. 7i). Further, increased expression of CCL5, CCL7, CXCL1 and IL13 correlated with presence of multifocal tumors instead of solitary tumors, suggestive of association with intrahepatic metastasis (FIG. 7j). We conclude that TWIST1 expression in HCC is associated with pro-TAM cytokines, multifocal tumors with worse clinical outcome.

We found that Twist1 expression, in a new transgenic mouse model of metastatic HCC (MYC/Twist), in many types of human cancers and specifically in human HCC is associated with a cytokinome that is associated with macrophage recruitment and repolarization, that we can show in our transgenic model is causally required to induce metastasis. We found that in mouse and human HCC, Twist1 cytokinome includes: CCL2, CCL5, CCL7, CXCL1 and IL13. We showed that both CCL2 and IL13 are necessary and sufficient to elicit metastasis in a transgenic mouse model of HCC. The neutralization of CCL2 and IL13 is synergistic in abrogating Twist1 driven metastasis, by inhibiting both macrophage recruitment and polarization. Importantly, transcriptional analysis of around 9500 human samples from 33 cancers showed that high expression of human TWIST1 is strongly associated with pro-TAM cytokine expression, M2 macrophage infiltration and predicts poor clinical outcome. Finally, a prospective human clinical study confirmed that the TWIST1 associated pro-TAM cytokines are overexpressed specifically in HCC and predict more advanced multifocal disease. The generalizability of our results from in vivo transgenic mouse model across a multitude of human cancers and in a clinical study of human patients with HCC, suggests we identified a common mechanism of tumor invasion and metastasis. Finally, our observations have therapeutic implications for predicting metastasis and treating metastasis by targeting combinations of cytokines, in particular CCL2 and IL13.

Our results are consistent with studies that have shown that Twist1 is involved in cancer metastasis and/or metastasis and invasion of HCC. Our transgenic model is the first to show that Twist1 can elicit metastasis in an autochthonous in vivo mouse model of HCC. Our hepatocyte-specific and conditional transgenic model of Twist1 enabled us to bypass some limitations of xenograft and transplantation model, thus enabling us to analyze the tumor microenvironment in an immunocompetent host. Because our model is Tet system regulated, we avoid effects of oncogene expression during embryogenesis. We show that Twist1 combined with MYC leads to rapid progression of HCC with >90% of the mice developing extrahepatic metastasis within 12-20 weeks of oncogene induction with similar organ tropism as human HCC thus allowing us to study specific stages of metastasis progression. The high penetrance of the phenotype and abbreviated time course make this model a highly attractive preclinical model for evaluating future therapeutics for metastatic liver cancer.

Twist1 is a transcription factor which is physiologically expressed only during embryonic development and is pathologically overexpressed in cancer cells. It is known to facilitate embryogenesis by multiple mechanisms like EMT, inhibiting apoptosis and maintaining stemness. Twist1 expressing cancer cells co-opt these cell-autonomous processes as they clearly bestow a pro-metastatic advantage. Since MYC also directly influence these cell autonomous functions, we did not find significant differences in EMT, apoptosis or invasiveness between MYC- and MYC/Twist1-HCC. Twist1 has been reported to have another important embryonic function, which is to repress inflammation in the microenvironment by regulating cytokine expression. Our results show that Twist1 overexpression in cancer cells is co opting this embryonic program to regulate cell migration and inflammation through the production of specific cytokines.

The striking macrophage infiltration and M2 polarization noted in Twist1 expressing tumors prompted us to explore the cancer cytokinome to look for paracrine factors. In a previous study on breast cancer cell lines, Twist1 was shown to promote macrophage recruitment via induction of CCL2 but the broad effects of Twist1 on the cytokinome or the specific function of Twist1 on macrophage recruitment vs polarization remained to be explored. We took an unbiased approach to identify tumor-derived cytokines and performed a multiplex ELISA to evaluate a set of 38 key cytokines in the conditioned media of the cancer cells. Five cytokines were identified that are known to be pro-TAM including- CCL2, CCL5, CCL7, CXCL1 and IL13. Our in vitro neutralization experiments show that CCL2, CCL5, CCL7 and CXCL1 play a significant role in Twist1-mediated TAM recruitment while CCL5 and IL13 promote Twist1-mediated TAM polarization. The observation that multiple cytokines effectuate equivalent changes in our system is not surprising given that cytokines are known to have overlapping and redundant functions. But it underscores the challenges in targeting the macrophage activation pathway in cancer, and indicates the need to simultaneously inhibit more than one cytokine to effectively inhibit cancer progression.

Among the five cytokines that mediate Twist1 induced macrophage recruitment and polarization, CCL2 and IL13 had the most pronounced in vitro effects. Hence, we further evaluated the role of CCL2 and IL13. In vivo treatment of non metastatic MYC-HCC with combination of CCL2 and IL13 phenocopied Twist1 expression, and was sufficient confer a metastatic phenotype by eliciting macrophage recruitment and polarization. Treatment of MYC-HCC bearing mice with CCL2 alone, or IL13 alone, did not elicit metastasis. CCL2 treatment was indeed able to recruit macrophages to the tumor but this did not result in metastatic progression in the absence of TAM polarizing cytokine IL13. Thus, both macrophage recruitment and polarization play essential and complementary roles in Twist1 induced metastasis. Consistent with these observations, combined neutralization of CCL2 and IL13 in MYC/Twist1-HCC bearing mice was synergistic and led to near-complete abrogation of metastasis. While the role of Twist1 in recruiting macrophages has been reported, its ability to induce M2 polarization of macrophages is novel and previously unidentified.

Lastly, our results have potential clinical implications. First, we found that TWIST1 expression in 9500 patients with 33 different types of human cancer predicted clinical outcome. Also, TWIST1 expression in these tumors is strongly correlated with greater infiltration of protumorigenic macrophages (TAM) and higher pro-TAM cytokine expression. Hence TWIST1 expression can be used a biomarker to stratify patients in clinical trials for macrophage inhibitors. Second, we demonstrated that in humans with HCC the pro-TAM network of cytokines CCL2, CCL5, CXCL1, and IL13, are elevated the serum of patients with multifocal more advanced HCC. Measurement of these cytokines may be prognostic in patients with HCC, as noninvasive biomarkers of advanced HCC. Third, we found that the inhibition of CCL2 and IL13 synergistically impeded metastasis. Targeting combinations of pro-TAM cytokines are more likely to be effective to impede local invasion and metastasis of HCC, as well as other human cancers. Clinical studies of CCL2 inhibition alone has not been successful. We suggest that stratifying patients based on cytokine expression is useful to personalize anti-cytokine targeted therapies.

We have identified a novel mechanism by which Twist1 promotes metastasis via modulation of the cancer cytokinome to recruit protumorigenic macrophages. Our findings highlight that Twist1 overexpression can mediate metastasis by restoring an embryonic program of paracrine cross-talk between cancer cells and macrophages. We note we found that systemic introduction of pro-TAM cytokines was sufficient to drive cancer progression in a completely paracrine manner, suggesting that metastasis can be conferred on otherwise dormant cancer cells. This raises the provocative possibility that circumstances that promote the generalized induction of pro-TAM cytokines could elicit metastasis. Our observations also suggest a new therapeutic vulnerability of Twist1 driven metastasis through the neutralization of cytokines. We note that multiple cytokines have to be neutralized and the precise complement may be individual tumor specific. Our study provides a strong rationale for pursuing multipronged therapeutic strategies for metastasis like directly targeting Twist1 with small molecule inhibitors, blocking macrophages recruitment, and inhibition of combination of cytokines like CCL2 and IL13 to treat liver and many other human cancers.

### Materials and Methods

Transgenic mice: Mouse Twist1 cDNA was PCR cloned into the bidirectional tetO7 vector S2f-IMCg at EcoRI and Notl sites, replacing the eGFP ORF. The resultant construct, Twist1-tetO7-luc²⁸, was sequenced, digested with Kpnl and Xmnl, and used for injection of FVB/N pronuclei by the Stanford Transgenic Facility. Founders were screened by genotyping using PCR. Founders were mated to LAP-tTA mice, and BLI was used to additionally screen for functional Twist1-tet07-luc founders, subsequently termed LAP-tTA/TRE-Twist1/Luc. The LAP-tTA, and TetO-MYC transgenic lines have been described previously ^{29,55,56.} LAP-tTA/TRE-Twist1/Luc mice were mated to LAP-tTA/TRE-MYC mice, and progeny were screened by PCR. Doxycycline (Dox- Sigma) was administered in the drinking water weekly at 0.1 mg/mL during mating and continuing until mice reached 6 weeks of age. Animals were euthanized upon disease morbidity as assessed by tumor burden. Macrometastases were assessed upon necropsy and tissues were collected and stored for further analysis. All procedures were performed in accordance with APLAC protocols and animals were housed in a pathogen-free environment.

Small animal imaging: in vivo bioluminescent imaging (BLI) was utilized to confirm oncogene activation in transgenic mice beginning one week before, and continuing each week following, Dox removal. BLI was performed on an IVIS Spectrum (Caliper Life Sciences, Hopkinton, MA). Briefly, mice were injected i.p. with the substrate D-Luciferin (150 mg/kg) and then anesthetized with 2.5% isoflurane delivered by the Xenogen XGI-8 5-port Gas Anesthesia System. Animals were then placed into the IVIS Spectrum, and Living Image Software was used to collect, archive, and analyze photon fluxes and transform them into pseudocolor images. MRI scans were performed using a 7 T small animal MRI scanner (Bruker Inc., Billerica, MA, Stanford Small Animal Imaging Facility, CA) equipped with a 40 mm Millipede RF coil (ExtendMR LLC, Milpitas, CA). Under anesthesia by inhalation of 1-3% isoflurane mixed in with medical-grade oxygen via nose-cone, and acquisitions were gated using the respiratory triggering. For tumor detection, a respiration triggered T2-weighted 3D turbo spin echo sequence was used (TR/ TE 3000/205 ms, voxel size (0.22 mm³). The isotropic voxel size of 0.22 mm in all directions provides a high in plane and across plane resolution. Thereby, the location of one tumor could be defined in all three orientations using specific landmarks, such as major vessels or other tumors. T2-weighted anatomical imaging was performed approximately once weekly. Anatomical and parametric images were analyzed and tumor volumes were measured using Osirix image processing software (Osirix, UCLA, and Los Angeles, CA).

Cell Culture: Conditional HCC cell lines were derived from LAP-tTA and TetO-MYC or -MYC/Twist1 mice. Cells were grown in DMEM (Invitrogen), supplemented with 10% FBS (Invitrogen), and cultured at 37° C. in a humidified incubator with 5% CO2. Cell lines were confirmed to be negative for mycoplasma contamination.

Orthotopic transplantation assay: An orthotopic mouse model was established by transplanting mouse MYC- or MYC/Twist1-HCC tumors (1 mm³) under the liver capsule of NOD/Scid/Gamma (NSG) recipient mice. Bioluminescent (BLI) and MRI scans monitoring are used to monitor tumor engraftment and growth. Mice are euthanized once predetermined specific endpoints are met or based on morbidity whichever occurs first.

Intravenous transplantation assay: An intravenous transplantation mouse model was established by tail vein intravenous injection of 500,000 MYC- or MYC/Twist1-HCC cells of NOD/Scid/Gamma (NSG) recipient mice. Bioluminescent (BLI) monitoring is used to monitor tumor metastasis in lungs. Mice are euthanized once predetermined specific endpoints are met or based on morbidity whichever occurs first.

RNA sequencing: RNA sequencing was performed at the Beijing Genomics Institute (BGI) using their BGIseq 500 platform single end 150 bp, 20 million reads per sample. Genes expression level is quantified by a software package called RSEM. We counted the number of identified expressed genes and calculated its proportion to total gene number in database for each sample RNA sequencing data are deposited in Gene Expression Omnibus (GEO). DEseq software was used to perform differential expression analysis. Ingenuity Pathway analysis was used to perform functional pathway analysis.

in vivo treatment mouse models: For antibody treatment, mice were injected i.p. with isotype control IgG or anti-CCL2 (BioXcell), -IL13 and -IL4 (Genentech) antibody (10 mg/kg body weight three times per week). For recombinant cytokines treatment, mice were injected i.p. three times per week with PBS or CCL2 (Peprotech, 500 ng/mouse), IL13 and IL4 (Peprotech, 250 ng/mouse). For clodronate liposomes (CL) treatment, CL or control liposomes were administered i.p. at 6.5 µl/g body weight 3 times per week to NSGs mice. CL and control treatments were administered to NSG mice previously injected i.v. with 0.5×10⁶ MYC/twist1-HCC cells or orthotopically transplanted with MYC/Twist1-HCC tumors. Experimental and control mice were killed 4 weeks after tumor were transplanted. Primary tumors and lung metastases were collected for H&E staining and IHC.

Immunohistochemistry and Immunofluorescence: Paraffin embedded tumor sections were deparaffinized by successive incubations in xylene, graded washes in ethanol, and deionized water. Epitope unmasking was performed by steaming in DAKO antigen retrieval solution for 45 minutes. Paraffin embedded sections were immunostained with MYC (1:150, Epitomics), or cleaved caspase 3 (1:100, Cell Signaling technology), phospho histone 3 (1:200, Cell Signaling Technology), F4/80 (1:50, ThermoFisher), CD4 (1 :1000, Abcam), Neutrophil (1:100, Abcam), E-cadherin (1:100, BD Pharmingen), or beta-catenin (1:100, BD Pharmingen) overnight at 4° C. The tissue was washed with PBS and incubated with biotinylated anti-rabbit, anti-rat or anti-mouse for 30 minutes at room temperature (1:300 Vectastain ABC kit, Vector Labs). Sections were developed using 3,3′- Diaminobenzidine (DAB, Vector Labs), counterstained with hematoxylin, and mounted with Permount. Images were obtained on a Philips Ultrafast Scanner.

Quantification of IHC staining analysis: Images are analyzed in Icy (Biolmage Analysis Unit, Paris). Areas of immunopositivity are selected for positive values in the Color Picker threshold tool in the support vector machine (SVM) tab. Unstained nuclei and cytoplasm areas are chosen for negative values. Default values are used for the kernel. Immunopositive areas are selected as regions of interest (ROIs). Subsequently, ROIs are separated. Those with interior size <68 pixels are eliminated from the ROI table as they correspond to small specks of non-specific immunopositivity, whereas larger areas corresponded closely to distinct positive cells. Counts were obtained from the ROI tables.

Wound Healing Assay: 1×10⁶ MYC- or MYC/Twist1-HCC cells per well were seeded in 6-well plate and cultured overnight in culture medium. Thereafter, a scratch (wound) was introduced in the confluent cell layer using a yellow tip placed in a scaffold, allowing standardization of the scratch. Cells were washed three times with PBS to remove detached cells. Cells were then incubated with supernatant of primary macrophages harvested from MYC- or MYC/Twist1-HCC primary tumors for 24 h. Pictures of a defined wound spot were made with a Leica DM16000 microscope at t = 0, 24, 48 and 72 h. The area of the wound in the microscopic pictures was measured using Image J software (National Institutes of Health, MD) at different time points. The percentage wound healing after 72 h was calculated in relative to the total wound area at t = 0 h of the same wound spot.

Transwell chamber migration assay: Cell migration was assessed using a 12 well transwell chamber with 8 µm filter inserts (Corning). Raw macrophages were seeded in the upper chamber and MYC/Twist1 cells were seeded in the lower chamber. Neutralizing antibody to CCL2, CCL5, CCL7, CXCL1, IL13 or IL4 were added to the lower chamber in triplicates. After 16 hours the migrated cells were fixed in 4% paraformaldehyde (PFA) and 100% methanol. The non-migrated cells were gently removed with a swab. Cells in the lower surface of the membrane with stained with 0.5% crystal violet for 20 mins. The membranes were images and number of macrophages in 10 random fields were counted. The experiment was performed in triplicates.

3D co-culture system of Macrophages and HCC cells. The 24-well plates were precoated with 10×10³ mouse Raw 264.7 macrophages (ATCC) resuspended in 200 ul of Matrigel growth factor reduced (Corning) for 30 mins at 37° C. Then, 80×10³ MYC or MYC/Twist1-HCC cells resuspended in 200 ul of Matrigel and directly seeded onto the 24-well plate precoated with matrigel+macrophage mixture. The cells were incubated at 37° C. for up to 1 week to allow the spheroids to form. Recombinant CCL2 (50 ng/ml) or IL13, IL4 (25 ng/ml each) and anti-CCL2 antibody (30 ug/ml), or anti-IL13 antibody, anti-IL4 antibody (20 µg/ml each) were added directly to the coculture and refreshed every 48 h. Organoids were fixed with 10% PFA overnight, paraffin embedded and sectioned (4-5 µm) as previously described. Sections were deparaffinized and stained with H&E for the initial histology analysis. For further immunohistochemistry analysis, we used F4/80 antibody as described above. All assays were performed at least 3 times.

Quantitative Real-Time PCR: RNA was isolated using RNeasy plus mini kit according to the manufacturer’s instructions (Qiagen). cDNA was synthesized using SuperScript III (ThermoFisher). qPCR was performed using specific primers (Supplemental Table 1) and SYBR Green (Roche) in an Applied Biosystems Real Time PCR System (Life Technologies). Data were normalized to UBC. A minimum of 3 biological and 3 technical replicates were used for all qPCR experiments.

Luminex - eBioscience/Affymetrix Magnetic bead Kits: This assay was performed in the Human Immune Monitoring Center at Stanford University. Human 62-plex or Mouse 38 plex kits were purchased from eBiosciences/Affymetrix and used according to the manufacturer’s recommendations with modifications as described below. Briefly: Beads were added to a 96 well plate and washed in a Biotek ELx405 washer. Samples were added to the plate containing the mixed antibody-linked beads and incubated at room temperature for 1 hour followed by overnight incubation at 4° C. with shaking. Cold and Room temperature incubation steps were performed on an orbital shaker at 500-600 rpm. Following the overnight incubation plates were washed in a Biotek ELx405 washer and then biotinylated detection antibody added for 75 minutes at room temperature with shaking. Plate was washed as above and streptavidin-PE was added. After incubation for 30 minutes at room temperature wash was performed as above and reading buffer was added to the wells. Each sample was measured in duplicate. Plates were read using a Luminex 200 instrument with a lower bound of 50 beads per sample per cytokine. Custom assay Control beads by Radix Biosolutions are added to all wells.

Immune cell deconvolution analysis: The CIBERSORT gene expression deconvolution package was used to estimate the immune cell composition in the MYC- and MYC/Twist1-HCC. The LM22 signature was used as the immune cell gene signature. We modified it for studying mouse immune subsets by carefully converting the genes in the signature to their respective mouse orthologs. The settings for the run were: 1000 permutation with quantile normalisation disabled. The student T-tests was used to infer the statistical significance of the predicted immune cell populations where P<0.05 was considered significant.

TCGA analysis: The pan cancer RNAseq data was downloaded from the GDC data portal on Oct. 15, 2018. Spearman test was used for correlation analysis. Kaplan Meier analysis was performed for survival analysis. K means clustering was used to stratify patients into two groups based on MYC and TWIST1 expression.

Statistics: Differences between groups were analyzed using Student’s t-test or one-way analysis of variance (ANOVA). A P value of less than 0.05 was considered to be significant and is indicated by one asterisk (*), a P value of less than 0.01 is indicated by two asterisks (**), a P value of less than 0.001 is indicated by three asterisks (***), and a Pvalue of less than 0.0001 is indicated by four asterisks (****). All graphs are presented as the mean +/- SEM. Analyses were

### References

Fidler, I. J. The pathogenesis of cancer metastasis: the ‘seed and soil’ hypothesis revisited. Nat. Rev. Cancer 3, 453-458 (2003).

Gupta, G. P. & Massague, J. Cancer Metastasis: Building a Framework. Cell 127, 679-695 (2006).

Lambert, A. W., Pattabiraman, D. R. & Weinberg, R. A. Emerging Biological Principles of Metastasis. Cell 168, 670-691 (2017).

Chaffer, C. L. & Weinberg, R. A. A Perspective on Cancer Cell Metastasis. Scienc331, 1559-1564 (2011).

Xu, Y. et al. Twist1 promotes breast cancer invasion and metastasis by silencing Foxa1 expression. Oncogene 36, 1157-1166 (2017).

Kim, Y. H. et al. TWIST1 and SNAI1 as markers of poor prognosis in human colorectal cancer are associated with the expression of ALDH1 and TGF-βL. Oncol. Rep. 31, 1380-1388 (2014).

da Silva, S. D. et al. TWIST1 is a molecular marker for a poor prognosis in oral cancer and represents a potential therapeutic target. Cancer 120, 352-362 (2014).

Wushou, A., Hou, J., Zhao, Y.-J. & Shao, Z.-M. Twist-1 up-regulation in carcinoma correlates to poor survival. Int. J. Mol. Sci. 15, 21621-21630 (2014).

Lee, T. K. et al. Twist overexpression correlates with hepatocellular carcinoma metastasis through induction of epithelial-mesenchymal transition. Clin. Cancer Res. 12, 5369-5376 (2006).

Lee, K.-W. et al. Twist1 is an independent prognostic factor of esophageal squamous cell carcinoma and associated with its epithelial-mesenchymal transition. Ann. Surg. Oncol. 19, 326-335 (2012).

Thisse, B., El Messal, M. & Perrin-Schmitt, F. The Twist Gene: isolation of a Drosophila zygotle gene necessary for the establishment of dorsoventral pattern. Nucleic Acids Res. 15, 3439-3453 (1987).

Spicer, D. B., Rhee, J., Cheung, W. L. & Lassar, A. B. Inhibition of myogenic bHLH and MEF2 transcription factors by the bHLH protein Twist. Science 272, 1476-1480 (1996).

Lee, M.-S, Lowe, G. N., Strong, D. D., Wergedal, J. E. & Glackin, C. A. TWIST, a basic helix-loop-helix transcription factor, can regulate the human osteogenic lineage. J. Cell. Biochem. 75, 566-577 (1999).

Soo, K. et al. Twist function is required for the morphogenesis of the cephalic neural tube and the differentiation of the cranial neural crest cells in the mouse embryo. Dev. Biol. 247, 251-270 (2002).

Chen, Z. F. & Behringer, R. R. twist is required in head mesenchyme for cranial neural tube morphogenesis. Genes Dev. 9, 686-699 (1995).

Maestro, R. et al. twist is a potential oncogene that inhibits apoptosis. Genes Dev. 13, 2207-2217 (1999).

Beck, B. et al. Different levels of Twist1 regulate skin tumor initiation, stemness, and progression. Cell Stem Cell 16, 67-79 (2015).

Qian, B.-Z. & Pollard, J. W. Macrophage diversity enhances tumor progression and metastasis. Cell 141, 39-51 (2010).

Wan, S. et al. Tumor-associated macrophages produce interleukin 6 and signal via STAT3 to promote expansion of human hepatocellular carcinoma stem cells. Gastroenterology 147, 1393-1404 (2014).

Deng, Y.-R., Liu, W.-B., Lian, Z.-X., Li, X. & Hou, X. Sorafenib inhibits macrophage-mediated epithelial-mesenchymal transition in hepatocellular carcinoma. Oncotarget 7, 38292-38305 (2016).

Lu, T. et al. Tumor-infiltrating myeloid cells induce tumor cell resistance to cytotoxic T cells in mice. J. Clin. Invest. 121, 4015-4029 (2011).

Kim, J. et al. IL-10 production in cutaneous basal and squamous cell carcinomas. A mechanism for evading the local T cell immune response. J. Immunol. 155, 2240-2247 (1995).

De, I. et al. CSF1 Overexpression Promotes High-Grade Glioma Formation without Impacting the Polarization Status of Glioma-Associated Microglia and Macrophages. Cancer Res. 76, 2552-2560 (2016).

Franklin, R. A. et al. The cellular and molecular origin of tumor-associated macrophages. Science 344, 921-925 (2014).

Šošić, D., Richardson, J. A., Yu, K., Ornitz, D. M. & Olson, E. N. Twist regulates cytokine gene expression through a negative feedback loop that represses NF-kappaB activity. Cell 112, 169-180 (2003).

Sharif, M. N. et al. Twist mediates suppression of inflammation by type I IFNs and Axl. J. Exp. Med. 203, 1891-1901 (2006).

Low-Marchelli, J. M. et al. Twist1 induces CCL2 and recruits macrophages to promote angiogenesis. Cancer Res. 73, 662-671 (2013).

Tran, P. T. et al. Twist1 suppresses senescence programs and thereby accelerates and maintains mutant Kras-induced lung tumorigenesis. PLoS Genet. 8, e1002650 (2012).

Shachaf, C. M. et al. MYC inactivation uncovers pluripotent differentiation and tumour dormancy in hepatocellular cancer. Nature 431, 1112-1117 (2004).

Newman, A. M. et al. Robust enumeration of cell subsets from tissue expression profiles. Nat. Methods 12, 453-457 (2015).

Moreno, S. G. Depleting Macrophages in vivo with Clodronate-Liposomes. Methods Mol. Biol. 1784, 259-262 (2018).

McWhorter, F. Y., Wang, T., Nguyen, P., Chung, T. & Liu, W. F. Modulation of macrophage phenotype by cell shape. Proc. Natl. Acad. Sci. U. S. A. 110, 17253-17258 (2013).

Tang, Z. et al. GEPIA: a web server for cancer and normal gene expression profiling and interactive analyses. Nucleic Acids Res. 45, W98-W102 (2017).

Ye, Q.-H. et al. Predicting hepatitis B virus-positive metastatic hepatocellular carcinomas using gene expression profiling and supervised machine learning. Nat. Med. 9, 416-423 (2003).

Thorsson, V. etal. The Immune Landscape of Cancer. Immunity 48, 812-830.e14 (2018).

Bisgaard, L. S. et al. Bone marrow-derived and peritoneal macrophages have different inflammatory response to oxLDL and M1/M2 marker expression - implications for atherosclerosis research. Sci. Rep. 6, (2016).

Meng, J. et al. Twist1 Regulates Vimentin through Cul2 Circular RNA to Promote EMT in Hepatocellular Carcinoma. Cancer Res. 78, 4150-4162 (2018).

Niu, R. F. et al. Up-regulation of Twist induces angiogenesis and correlates with metastasis in hepatocellular carcinoma. J. Exp. Clin. Cancer Res. 26, 385-394 (2007).

Yang, M.-H. et al. Comprehensive analysis of the independent effect of twist and snail in promoting metastasis of hepatocellular carcinoma. Hepatology 50, 1464-1474 (2009).

Gómez-Cuadrado, L., Tracey, N., Ma, R., Qian, B. & Brunton, V. G. Mouse models of metastasis: progress and prospects. Dis. Model. Mech. 10, 1061-1074 (2017).

Vincentz, J. W. et al. An absence of Twist1 results in aberrant cardiac neural crest morphogenesis. Dev. Biol. 320, 131-139 (2008).

Hebrok, M., Wertz, K. & Füchtbauer, E.-M. M-twist Is an Inhibitor of Muscle Differentiation. Dev. Biol. 165, 537-544 (1994).

Yang, J. et al. Twist, a Master Regulator of Morphogenesis, Plays an Essential Role in Tumor Metastasis. Cell 117, 927-939 (2004).

Morel, A.-P. et al. Generation of Breast Cancer Stem Cells through Epithelial-Mesenchymal Transition. PLoS One 3, e2888 (2008).

Cho, K. B., Cho, M. K., Lee, W. Y. & Kang, K. W. Overexpression of c-myc induces epithelial mesenchymal transition in mammary epithelial cells. Cancer Lett. 293, 230-239 (2010).

Yin, S., Cheryan, V. T., Xu, L., Rishi, A. K. & Reddy, K. B. Myc mediates cancer stem-like cells and EMT changes in triple negative breast cancers cells. PLoS One 12, e0183578 (2017).

Tsai, J. H., Donaher, J. L., Murphy, D. A., Chau, S. & Yang, J. Spatiotemporal regulation of epithelial-mesenchymal transition is essential for squamous cell carcinoma metastasis. Cancer Cell 22, 725-736 (2012).

Ozaki, K. & Leonard, W. J. Cytokine and cytokine receptor pleiotropy and redundancy. J. Biol. Chem. 277, 29355-29358 (2002).

Lim, S. Y., Yuzhalin, A. E., Gordon-Weeks, A. N. & Muschel, R. J. Targeting the CCL2-CCR2 signaling axis in cancer metastasis. Oncotarget7, 28697-28710 (2016).

Pienta, K. J. et al. Phase 2 study of carlumab (CNTO 888), a human monoclonal antibody against CC-chemokine ligand 2 (CCL2), in metastatic castration-resistant prostate cancer. Invest. New Drugs 31, 760-768 (2013).

Brana, I. et al. Carlumab, an anti-C-C chemokine ligand 2 monoclonal antibody, in combination with four chemotherapy regimens for the treatment of patients with solid tumors: an open-label, multicenter phase 1b study. Target. Oncol. 10, 111-123 (2015).

Yochum, Z. A. etal. A First-in-Class TWIST1 Inhibitor with Activity in Oncogene-Driven Lung Cancer. Mol. Cancer Res. 15, 1764-1776 (2017).

Cannarile, M. A. et al. Colony-stimulating factor 1 receptor (CSF1 R) inhibitors in cancer therapy. J Immunother Cancer 5, 53 (2017).

Vela, M., Aris, M., Llorente, M., Garcia-Sanz, J. A. & Kremer, L. Chemokine receptor-specific antibodies in cancer immunotherapy: achievements and challenges. Front. Immunol. 6, 12 (2015).

Felsher, D. W. & Michael Bishop, J. Reversible Tumorigenesis by MYC in Hematopoietic Lineages. Mol. Cell 4, 199-207 (1999).

Kistner, A. et al. Doxycycline-mediated quantitative and tissue-specific control of gene expression in transgenic mice. Proc. Natl. Acad. Sci. U. S. A. 93, 10933-10938 (1996).

Ootani, A. et al. Sustained in vitro intestinal epithelial culture within a Wnt-dependent stem cell niche. Nat. Med. 15, 701-706 (2009).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

What is claimed is:

1. A method of treating, reducing, or preventing the metastasis or invasion of a cancer in a mammalian patient, the method comprising:
administering to the patient an effective dose of a combination of (i) an inhibitor of a cytokine that induces recruitment of macrophages to a tumor environment and (ii) an inhibitor of a cytokine that polarizes macrophages to an M2 phenotype, in a dose effective to decrease metastasis of the cancer.

2. The method of claim 1, wherein the inhibitor (i) inhibits activity of one or more of CCL2, CCL5, CCL7 and CXCL1.

3. The method of claim 1 or claim 2, wherein the inhibitor (i) inhibits CCL2.

4. The method of claim 3, wherein the inhibitor of CCL2 is an antibody.

5. The method of any of claims 1-4, wherein the inhibitor (ii) inhibits IL-13 activity.

6. The method of claim 5, wherein the inhibitor of IL-13 is an antibody.

7. The method of any of claims 1-6, wherein the cancer is a solid cancer.

8. The method of claim 7, wherein the solid cancer is a carcinoma.

9. The method of claim 8, wherein the cancer is a hepatocellular carcinoma.

10. The method of any of claims 1-9, wherein the combination of agents provides for a synergistic reduction in metastasis of the cancer.

11. The method of any of claims 1-10, further comprising testing the cancer for over-expression of Twist1, wherein a patient selected for therapy comprises a cancer that overexpresses Twist1.

12. The method of any of claims 1-10, further comprising testing the cancer for expression of a cytokinome associated with Twist1, wherein a patient selected for therapy comprises a cancer that expresses a Twist1 associated cytokinome.

13. The method according to any of claims 1-12, wherein the patient is a mouse.

14. The method according to any of claims 1-12, wherein the patient is a human.

15. The method of any of claims 1-14, wherein the agents are administered concomitantly.

16. A kit for use in the methods of any of claims 1-15.

17. An autochthonous transgenic mouse model for cancer, wherein the animal comprises a transgene of (i) an oncogene; and (ii) a metastasis promoting protein, each operably linked to a tet resonsive promoter element (TRE) promoter; wherein the animal expresses tetracycline-controlled transactivator protein (tTA) driven by a tissue specific promoter.

18. The transgenic animal of claim 1, wherein the oncogene is MYC and and the metasis promoting protein is TWIST1.

19. The transgenic animal of claim 17 or claim 18, wherein the tissue specific promoter is a liver specific promoter.

20. The transgenic animal of claim 19, wherein the liver specific promoter is liver-enriched activator protein (LAP) promoter.
